(19) United States
(12) Patent Application Publication
(10) Pub. No.: US 2025/0099331 A1
(43) Pub. Date: Mar. 27, 2025

(54) MEDICAL DEVICE SYSTEM AND METHOD FOR PREPARING A DOSE

(71) Applicant: BLUEROCK THERAPEUTICS LP, Cambridge, MA (US)

(72) Inventors: Peter ALDRIDGE, Cambridge, MA (US); Philippe MARCHAND, Cambridge, MA (US); Ibrahim DOMIAN, Cambridge, MA (US); Anna M GALEA, Cambridge, MA (US); Gregory POLLIERI, Cambridge, MA (US); Angel Leonardo GUERRERO PALACIO, Cambridge, MA (US); Brian MAC GIOLLA RI, Cambridge, MA (US); Jonathan RUBIANTO, Cambridge, MA (US); Matthew Gardner MAHER, Cambridge, MA (US); Michael SCOTT, Cambridge, MA (US)

(73) Assignee: BLUEROCK THERAPEUTICS LP, Cambridge, MA (US)

(21) Appl. No.: 18/725,451

(22) PCT Filed: Dec. 27, 2022
(86) PCT No.: PCT/US2022/054102

§ 371 Date: Jun. 28, 2024

Related U.S. Application Data
(60) Provisional application No. 63/294,851, filed on Dec. 30, 2021.
(60) Provisional application No. 63/294,853, filed on Dec. 30, 2021.
(60) Provisional application No. 63/294,854, filed on Dec. 30, 2021.
(60) Provisional application No. 63/435,196, filed on Dec. 23, 2022.

(51) Int. Cl. A61J 1/20

(52) U.S. Cl. CPC A61J 1/2037; A61J 1/2051; A61J 1/2075; A61J 1/2082; A61J 1/2089

(57) ABSTRACT

A medical device system including a first container and a second container, the first container including a first container outer body defining a first interior space connected to a first opening of the first container; and a first container-side valve configured to be activated, upon connection of the second container to the first container, from a normally closed position into the first interior space to an activated position, and the second container including a second container outer body defining a second container interior space and a stem fluidly connected to the second container interior space, wherein, upon connection of the second container to the first container, the first container-side valve is configured to be brought into contact with the stem and the stem is configured to activate the first container-side valve from the normally closed position to the activated position.

### CROSS-REFERENCE TO RELATED APPLICATIONS

This application claims priority to U.S. Provisional Application No. U.S. 63/294,851, filed on Dec. 30, 2021, U.S. Provisional Application No. U.S. 63/294,853, filed on Dec. 30, 2021, U.S. Provisional Application No. U.S. 63/294,854, filed on Dec. 30, 2021, and U.S. Provisional Application No. U.S. 63/435,196, filed on Dec. 23, 2022, the entire contents of each of which are incorporated herein by reference.

### BACKGROUND

The present disclosure relates in general to a medical device system and a method for preparing a dose of a formulated drug product. The formulated drug product can include a cell-based formulated drug product.

A need has been identified to provide improvements to the process for preparing doses of the cell-based formulated drug product. The improvements include, but are not limited to, preparing doses of the formulated drug product with a consistent volume and a cell concentration that meets a predetermined target concentration, and minimizing or eliminating a risk of contamination of the formulated drug product in different environments such as an operating room or an off-site location away from the operating room without access to a clean environment such as a biosafety cabinet or a cleanroom.

### SUMMARY

In a first embodiment, a medical device system is described. The medical device system comprises: a first container; and a second container, wherein the first container comprises: a first container outer body, wherein the first container outer body defines a first interior space connected to a first opening of the first container; and a first container-side valve configured to be activated, upon connection of the second container to the first container, from a normally closed position that relatively reduces flow through the first opening of the first container into the first interior space to an activated position that relatively increases flow through the first opening of the first container into the first interior space, and wherein the second container comprises: a second container outer body that defines a second container interior space; and a stem fluidly connected to the second container interior space, wherein, upon connection of the second container to the first container, the first container-side valve is configured to be brought into contact with the stem and the stem is configured to activate the first container-side valve from the normally closed position to the activated position.

In the first embodiment, the second container interior space extends along a longitudinal direction, at a first end portion of the second container interior space along the longitudinal direction, a width of the second container interior space decreases towards a first end opening of the second container interior space, and the stem is fluidly connected to the first end opening of the second container interior space and extends along the longitudinal direction.

In the first embodiment, the stem is connected to the second container outer body to be stationary relative to the second container interior space.

In the first embodiment, the first container further comprises a sheath defining a sheath interior space, and at least a portion of the first container outer body is configured to be removably inserted within the sheath interior space.

In the first embodiment, the first container-side valve is configured to be connected to a first end portion of the sheath, and the first container outer body is configured to be arranged within the sheath interior space to contact the first container-side valve such that upon activation of the first container-side valve from the normally closed position to the activated position the flow through the first opening of the first container into the first interior space is relatively increased.

In the first embodiment, the first container further comprises a sheath cap configured to be removably connected to a second end portion of the sheath with the first container outer body being enclosed within the sheath by the sheath cap.

In the first embodiment, the sheath cap comprises: a vent configured to vent air displaced from first interior space; and a filter arranged within the vent.

In the first embodiment, the first container further comprises a plunger seal arranged within the first interior space of the first container outer body, wherein the plunger seal is configured to receive a force to move the plunger seal within the first interior space to change a volume of space within the first interior space between the plunger seal and the first container-side valve.

In the first embodiment, the sheath cap defines a through hole through which gas in the interior space of the first container outer body is vented.

In the first embodiment, the medical device system further comprises a plunger, configured to be removably connected to the plunger seal.

In the first embodiment, the first container further comprises a plunger seal arranged within the first interior space, wherein the plunger seal is configured to receive a force to move the plunger seal within the first interior space to change a volume of space within the first interior space between the plunger seal and the first container-side valve.

In the first embodiment, the medical device system further comprises: a plunger configured to apply the force to the plunger seal to move the plunger seal within the first interior space to change the volume of space within the first interior space between the plunger seal and the first container-side valve.

In the first embodiment, the medical device system according further comprises: a movable body arranged within the first interior space of the first container outer body, the movable body is configured to move within the first interior space between the plunger seal and the first container-side valve.

In the first embodiment, the movable body comprises a stainless-steel body.

In the first embodiment, the movable body is configured to be moved within the first interior space by a magnetic force exerted from outside of the first container.

In the first embodiment, the medical device system further comprises:
a movable body arranged within the first interior space of the first container outer body and configured to move within the first interior space.

In the first embodiment, the movable body defines one or more through holes connecting a first side of the movable body facing the first container-side valve and a second side of the movable body opposite to the first side of the movable body.

In the first embodiment, the first container further comprises a valve cap configured to be removably connected to the first container-side valve to prevent direct contact of the first container-side valve from an exterior of the valve cap.

In a second embodiment, a medical device system is described. The medical device system comprises: a first container; and a second container, wherein the first container comprises: a first container outer body, wherein the first container outer body defines a first interior space connected to a first opening of the first container; a first container-side valve configured to be activated, upon connection of the second container to the first container, from a normally closed position that relatively reduces flow through the first opening of the first container into the first interior space to an activated position that relatively increases flow through the first opening of the first container into the first interior space; a sheath defining a sheath interior space, wherein at least a portion of the first container outer body is configured to be removably inserted within the sheath interior space; a sheath cap configured to be removably connected to a second end portion of the sheath with the first container outer body being enclosed within the sheath by the sheath cap; and optionally, a plunger seal arranged within the first interior space, wherein the plunger seal is configured to receive a force to move the plunger seal within the first interior space to change a volume of space within the first interior space between the plunger seal and the first container-side valve, and wherein the second container comprises: a second container outer body that defines a second container interior space; and a stem fluidly connected to the second container interior space, wherein, upon connection of the second container to the first container, the first container-side valve is configured to be brought into contact with the stem and the stem is configured to activate the first container-side valve from the normally closed position to the activated position.

In a third embodiment, a medical device system is described. The medical device system comprises: a first container; and a second container, wherein the first container comprises: a first container outer body, wherein the first container outer body defines a first interior space connected to a first opening of the first container; a first container-side valve configured to be activated, upon connection of the second container to the first container, from a normally closed position that relatively reduces flow through the first opening of the first container into the first interior space to an activated position that relatively increases flow through the first opening of the first container into the first interior space; and a sheath defining a sheath interior space, wherein at least a portion of the first container outer body is configured to be removably inserted within the sheath interior space, and wherein an exterior surface of the sheath comprises a first connection structure, and wherein the second container comprises: a second container outer body that defines a second container interior space; and a stem fluidly connected to the second container interior space, wherein the second container outer body comprises a second connection structure configured to be connected to the first connection structure of the sheath to connect the second container to the first container, and wherein, upon connection of the second container to the first container, the first container-side valve is configured to be brought into contact with the stem and the stem is configured to activate the first container-side valve from the normally closed position to the activated position.

In a fourth embodiment, a medical device system is described. The medical device system comprises: a first container; and a second container, wherein the first container comprises: a first container outer body, wherein the first container outer body defines a first interior space connected to a first opening of the first container; a first container-side valve configured to be activated, upon connection of the second container to the first container, from a normally closed position that relatively reduces flow through the first opening of the first container into the first interior space to an activated position that relatively increases flow through the first opening of the first container into the first interior space; and optionally, a plunger seal arranged within the first interior space, wherein the plunger seal is configured to receive a force to move the plunger seal within the first interior space to change a volume of space within the first interior space between the plunger seal and the first container-side valve, and wherein the second container comprises: a second container outer body that defines a second container interior space; and a stem fluidly connected to the second container interior space, wherein, upon connection of the second container to the first container, the first container-side valve is configured to be brought into contact with the stem and the stem is configured to activate the first container-side valve from the normally closed position to the activated position.

In a fifth embodiment, an apparatus is described. The apparatus comprises: a first container comprising: a first container outer body; a first container barrel, arranged within the first container outer body, that defines a first interior space connected to a first opening of the first container; and a first container-side valve configured to be activated, upon connection of a second container to the first container outer body, from a normally closed position that relatively reduces flow through the first opening of the first container into the first interior space to an activated position that relatively increases flow through the first opening of the first container into the first interior space.

In the fifth embodiment, the second container comprises a second container-side valve, and upon connection of the second container to the first container outer body, the first container-side valve is brought into contact with the second container-side valve.

In the fifth embodiment, upon connection of the second container to the first container outer body, the first container-side valve is configured to be contacted by and activated by the second container-side valve from the normally closed position to the activated position.

In the fifth embodiment, the second container comprises: a second container outer body that defines a second container barrel interior space connected a first opening of the second container, the second container-side valve is configured to be activated, upon connection of the second container to the first container outer body, from a normally closed position that relatively reduces flow through the first opening of the second container, from a normally closed position that relatively reduces flow through the first opening of the second container into the second container barrel interior space to an activated position that relatively increases flow through the first opening of the second container into the second container barrel interior space, and upon connection of the second container to the first container outer body, the first container-side valve is configured to be contacted by and activated by the second container-side valve from the normally closed position to the activated position and the second container-side valve is configured to be activated from the normally closed position to the activated position to communicate the second container barrel interior space with the first interior space.

In the fifth embodiment, the second container-side valve comprises a valve stem, and upon connection of the second container to the first container outer body, the first container-side valve is configured to be contacted by and activated by the valve stem from the normally closed position to the activated position and the second container-side valve is configured to be activated from the normally closed position to the activated position to communicate the second container barrel interior space with the first interior space through the valve stem.

In a sixth embodiment, a medical device system is described. The medical device system comprises: a first container; and a second container, wherein the first container comprises: a first container outer body; a first container barrel, arranged within the first container outer body, that defines a first interior space connected to a first opening of the first container; and a first container-side valve configured to be activated, upon connection of the second container to the first container outer body, from a normally closed position that relatively reduces flow through the first opening of the first container into the first interior space to an activated position that relatively increases flow through the first opening of the first container into the first interior space.

In the sixth embodiment, the second container comprises a second container-side valve, and upon connection of the second container to the first container outer body, the first container-side valve is brought into contact with the second container-side valve.

In the sixth embodiment, upon connection of the second container to the first container outer body, the first container-side valve is configured to be contacted by and activated by the second container-side valve from the normally closed position to the activated position.

In the sixth embodiment, the second container comprises: a second container outer body that defines a second container barrel interior space connected a first opening of the second container, the second container-side valve is configured to be activated, upon connection of the second container to the first container outer body, from a normally closed position that relatively reduces flow through the first opening of the second container, from a normally closed position that relatively reduces flow through the first opening of the second container from the second container barrel interior space to an activated position that relatively increases flow through the first opening of the second container from the second container barrel interior space, and upon connection of the second container to the first container outer body, the first container-side valve is configured to be contacted by and activated by the second container-side valve from the normally closed position to the activated position and the second container-side valve is configured to be activated from the normally closed position to the activated position to communicate the second container barrel interior space with the first interior space.

In the sixth embodiment, the second container-side valve comprises a valve stem, and upon connection of the second container to the first container outer body, the first container-side valve is configured to be contacted by and activated by the valve stem from the normally closed position to the activated position and the second container-side valve is configured to be activated from the normally closed position to the activated position to communicate the second container barrel interior space with the first interior space through the valve stem.

In a seventh embodiment, a process for operating a medical device system is described. The process comprises: a first container; and a second container, wherein the first container comprises: a first container outer body; a first container barrel, arranged within the first container outer body, that defines a first interior space connected to a first opening of the first container; and a first container-side valve, and wherein the process comprises: aligning the first container and the second container; and connecting the second container to the first container outer body, wherein the first container-side valve is configured to be activated, upon connection of the second container to the first container outer body, from a normally closed position that relatively reduces flow through the first opening of the first container into the first interior space to an activated position that relatively increases flow through the first opening of the first container into the first interior space.

In the seventh embodiment, the second container comprises a second container-side valve, and the process comprises: in connecting the second container to the first container outer body, bringing the first container-side valve into contact with the second container-side valve.

In the seventh embodiment, the process comprises: in bringing the first container-side valve into contact with the second container-side valve, activating, by the second container-side valve, the first container-side valve from the normally closed position to the activated position.

In the seventh embodiment, the second container comprises: a second container outer body that defines a second container barrel interior space connected a first opening of the second container, and the process comprises: activating the second container-side valve, upon connection of the second container to the first container outer body, from a normally closed position that relatively reduces flow through the first opening of the second container, from a normally closed position that relatively reduces flow through the first opening of the second container from the second container barrel interior space to an activated position that relatively increases flow through the first opening of the second container from the second container barrel interior space, to communicate the second container barrel interior space with the first interior space.

In the seventh embodiment, the second container-side valve comprises a valve stem, and the process comprises, upon connection of the second container to the first container outer body: bringing the valve stem into contact with the first container-side valve to activate the first container-side valve from the normally closed position to the activated position and the second container-side valve from the normally closed position to the activated position to communicate the second container barrel interior space with the first interior space through the valve stem.

In the seventh embodiment, the process further comprising: pre-loading the first interior space of the first container with a cell delivery solution.

In the seventh embodiment, the process further comprising: loading a mixture comprising a plurality of cells and a wash solution in the second container barrel interior space.

In the seventh embodiment, the process further comprising: after communicating the second container barrel interior space with the first interior space, applying a centrifugal force to the medical device system to move the plurality of cells from the second container barrel interior space through the second container-side valve in the activated position and the first container-side valve in the activated position into the first interior space.

In an eighth embodiment, an apparatus is described. The apparatus comprises: a first container comprising: a first container outer body; a first container barrel, arranged within the first container outer body, that defines a first interior space connected to a first opening of the first container; and a first container-side valve configured to be activated, upon connection of a second container to the first container outer body, from a normally closed position that relatively reduces flow through the first opening of the first container into the first interior space to an activated position that relatively increases flow through the first opening of the first container into the first interior space; and a movable body arranged within the first interior space of the first container barrel, wherein the movable body is configured to be moved within the first interior space by a force exerted from outside of the first container.

In the eighth embodiment, the movable body comprises a magnet configured to be moved within the first interior space by a magnetic force exerted from outside of the first container.

In the eighth embodiment, the movable body defines one or more through-holes extending between a first surface of the movable body and a second surface of the movable body opposing the first surface.

In the eighth embodiment, the second container comprises a second container-side valve, and upon connection of the second container to the first container outer body, the first container-side valve is brought into contact with the second container-side valve.

In the eighth embodiment, upon connection of the second container to the first container outer body, the first container-side valve is configured to be contacted by and activated by the second container-side valve from the normally closed position to the activated position.

In the eighth embodiment, the second container comprises: a second container outer body that defines a second container barrel interior space connected a first opening of the second container, the second container-side valve is configured to be activated, upon connection of the second container to the first container outer body, from a normally closed position that relatively reduces flow through the first opening of the second container, from a normally closed position that relatively reduces flow through the first opening of the second container into the second container barrel interior space to an activated position that relatively increases flow through the first opening of the second container into the second container barrel interior space, and upon connection of the second container to the first container outer body, the first container-side valve is configured to be contacted by and activated by the second container-side valve from the normally closed position to the activated position and the second container-side valve is configured to be activated from the normally closed position to the activated position to communicate the second container barrel interior space with the first interior space.

In the eighth embodiment, the second container-side valve comprises a valve stem, and upon connection of the second container to the first container outer body, the first container-side valve is configured to be contacted by and activated by the valve stem from the normally closed position to the activated position and the second container-side valve is configured to be activated from the normally closed position to the activated position to communicate the second container barrel interior space with the first interior space through the valve stem.

In a ninth embodiment, a medical device system is described. The medical device system comprises: a first container; and a second container, wherein the first container comprises: a first container outer body; a first container barrel, arranged within the first container outer body, that defines a first interior space connected to a first opening of the first container; a first container-side valve configured to be activated, upon connection of the second container to the first container outer body, from a normally closed position that relatively reduces flow through the first opening of the first container into the first interior space to an activated position that relatively increases flow through the first opening of the first container into the first interior space; and a movable body arranged within the first interior space of the first container barrel, wherein the movable body is configured to be moved within the first interior space by a force exerted from outside of the first container.

In the ninth embodiment, the medical device system further comprises: a first container mixing module comprising an external magnet, wherein the movable body comprises a magnet configured to be moved within the first interior space by a magnetic force exerted by the external magnet of the first container mixing module.

In the ninth embodiment, the movable body defines one or more through-holes extending between a first surface of the movable body and a second surface of the movable body opposing the first surface.

In the ninth embodiment, the second container comprises a second container-side valve, and upon connection of the second container to the first container outer body, the first container-side valve is brought into contact with the second container-side valve.

In the ninth embodiment, upon connection of the second container to the first container outer body, the first container-side valve is configured to be contacted by and activated by the second container-side valve from the normally closed position to the activated position.

In the ninth embodiment, the second container comprises: a second container outer body that defines a second container barrel interior space connected a first opening of the second container, the second container-side valve is configured to be activated, upon connection of the second container to the first container outer body, from a normally closed position that relatively reduces flow through the first opening of the second container, from a normally closed position that relatively reduces flow through the first opening of the second container from the second container barrel interior space to an activated position that relatively increases flow through the first opening of the second container from the second container barrel interior space, and upon connection of the second container to the first container outer body, the first container-side valve is configured to be contacted by and activated by the second container-side valve from the normally closed position to the activated position and the second container-side valve is configured to be activated from the normally closed position to the activated position to communicate the second container barrel interior space with the first interior space.

In the ninth embodiment, the second container-side valve comprises a valve stem, and upon connection of the second container to the first container outer body, the first container-side valve is configured to be contacted by and activated by the valve stem from the normally closed position to the activated position and the second container-side valve is configured to be activated from the normally closed position to the activated position to communicate the second container barrel interior space with the first interior space through the valve stem.

In a tenth embodiment, a process for operating a medical device system is described. The process comprises: a first container; and a second container, wherein the first container comprises: a first container outer body; a first container barrel, arranged within the first container outer body, that defines a first interior space connected to a first opening of the first container; a first container-side valve; and a movable body arranged within the first interior space of the first container barrel, and wherein the process comprises: aligning the first container and the second container; connecting the second container to the first container outer body, wherein the first container-side valve is configured to be activated, upon connection of the second container to the first container outer body, from a normally closed position that relatively reduces flow through the first opening of the first container into the first interior space to an activated position that relatively increases flow through the first opening of the first container into the first interior space; and exerting a force on the movable body from outside the first container to move the movable body within the first interior space.

In the tenth embodiment, the movable body comprises a magnet, and exerting the force on the movable body comprises exerting a magnetic force on the magnet of the movable body from outside the first container to move the movable body within the first interior space.

In the tenth embodiment, the movable body defines one or more through-holes extending between a first surface of the movable body and a second surface of the movable body opposing the first surface.

In the tenth embodiment, the second container comprises a second container-side valve, and the process comprises: in connecting the second container to the first container outer body, bringing the first container-side valve into contact with the second container-side valve.

In the tenth embodiment, the process comprises: in bringing the first container-side valve into contact with the second container-side valve, activating, by the second container-side valve, the first container-side valve from the normally closed position to the activated position.

In the tenth embodiment, the second container comprises: a second container outer body that defines a second container barrel interior space connected a first opening of the second container, and the process comprises: activating the second container-side valve, upon connection of the second container to the first container outer body, from a normally closed position that relatively reduces flow through the first opening of the second container, from a normally closed position that relatively reduces flow through the first opening of the second container from the second container barrel interior space to an activated position that relatively increases flow through the first opening of the second container from the second container barrel interior space, to communicate the second container barrel interior space with the first interior space.

In the tenth embodiment, the second container-side valve comprises a valve stem, and the process comprises, upon connection of the second container to the first container outer body: bringing the valve stem into contact with the first container-side valve to activate the first container-side valve from the normally closed position to the activated position and the second container-side valve from the normally closed position to the activated position to communicate the second container barrel interior space with the first interior space through the valve stem.

In the tenth embodiment, the process further comprises: pre-loading the first interior space of the first container with a cell delivery solution.

In the tenth embodiment, the process further comprises: loading a mixture comprising a plurality of cells and a wash solution in the second container barrel interior space.

In the tenth embodiment, the process further comprises: after communicating the second container barrel interior space with the first interior space, applying a centrifugal force to the medical device system to move the plurality of cells from the second container barrel interior space through the second container-side valve in the activated position and the first container-side valve in the activated position into the first interior space.

In an eleventh embodiment, an apparatus is described. The apparatus comprises: a first container comprising: a first container outer body; a first container barrel, arranged within the first container outer body, that defines a first interior space connected to a first opening of the first container and a second opening of the first container; and a first container-side valve configured to be activated, upon connection of a second container to the first container outer body, from a normally closed position that relatively reduces flow through the first opening of the first container into the first interior space to an activated position that relatively increases flow through the first opening of the first container into the first interior space; and a plunger seal arranged within the first container barrel between the first opening and the second opening, wherein the plunger seal is configured to receive a force to move the plunger seal within the first container barrel to change a volume of space within the first interior space between the first opening and the plunger seal.

In the eleventh embodiment, the apparatus further comprises: a plunger configured to be removably connected to the plunger seal and to transfer the force to move the plunger seal within the first container barrel to change the volume of space within the first interior space between the first opening and the plunger seal.

In the eleventh embodiment, the second container comprises a second container-side valve, and upon connection of the second container to the first container outer body, the first container-side valve is brought into contact with the second container-side valve.

In the eleventh embodiment, upon connection of the second container to the first container outer body, the first container-side valve is configured to be contacted by and activated by the second container-side valve from the normally closed position to the activated position.

In the eleventh embodiment, the second container comprises: a second container outer body that defines a second container barrel interior space connected a first opening of the second container, the second container-side valve is configured to be activated, upon connection of the second container to the first container outer body, from a normally closed position that relatively reduces flow through the first opening of the second container, from a normally closed position that relatively reduces flow through the first opening of the second container into the second container barrel interior space to an activated position that relatively increases flow through the first opening of the second container into the second container barrel interior space, and upon connection of the second container to the first container outer body, the first container-side valve is configured to be contacted by and activated by the second container-side valve from the normally closed position to the activated position and the second container-side valve is configured to be activated from the normally closed position to the activated position to communicate the second container barrel interior space with the first interior space.

In the eleventh embodiment, the second container-side valve comprises a valve stem, and upon connection of the second container to the first container outer body, the first container-side valve is configured to be contacted by and activated by the valve stem from the normally closed position to the activated position and the second container-side valve is configured to be activated from the normally closed position to the activated position to communicate the second container barrel interior space with the first interior space through the valve stem.

In a twelfth embodiment, a medical device system is described. The medical device system comprises: a first container; and a second container, wherein the first container comprises: a first container outer body; a first container barrel, arranged within the first container outer body, that defines a first interior space connected to a first opening of the first container and a second opening of the first container; a first container-side valve configured to be activated, upon connection of the second container to the first container outer body, from a normally closed position that relatively reduces flow through the first opening of the first container into the first interior space to an activated position that relatively increases flow through the first opening of the first container into the first interior space; and a plunger seal arranged within the first container barrel between the first opening and the second opening, wherein the plunger seal is configured to receive a force to move the plunger seal within the first container barrel to change a volume of space within the first interior space between the first opening and the plunger seal.

In the twelfth embodiment, the medical device system further comprises: a plunger configured to be removably connected to the plunger seal and to transfer the force to move the plunger seal within the first container barrel to change the volume of space within the first interior space between the first opening and the plunger seal.

In the twelfth embodiment, the second container comprises a second container-side valve, and upon connection of the second container to the first container outer body, the first container-side valve is brought into contact with the second container-side valve.

In the twelfth embodiment, upon connection of the second container to the first container outer body, the first container-side valve is configured to be contacted by and activated by the second container-side valve from the normally closed position to the activated position.

In the twelfth embodiment, the second container comprises: a second container outer body that defines a second container barrel interior space connected a first opening of the second container, the second container-side valve is configured to be activated, upon connection of the second container to the first container outer body, from a normally closed position that relatively reduces flow through the first opening of the second container, from a normally closed position that relatively reduces flow through the first opening of the second container from the second container barrel interior space to an activated position that relatively increases flow through the first opening of the second container from the second container barrel interior space, and upon connection of the second container to the first container outer body, the first container-side valve is configured to be contacted by and activated by the second container-side valve from the normally closed position to the activated position and the second container-side valve is configured to be activated from the normally closed position to the activated position to communicate the second container barrel interior space with the first interior space.

In the twelfth embodiment, the second container-side valve comprises a valve stem, and upon connection of the second container to the first container outer body, the first container-side valve is configured to be contacted by and activated by the valve stem from the normally closed position to the activated position and the second container-side valve is configured to be activated from the normally closed position to the activated position to communicate the second container barrel interior space with the first interior space through the valve stem.

In a thirteenth embodiment, a process for operating a medical device system is described. The process comprises: a first container; and a second container, wherein the first container comprises: a first container outer body; a first container barrel, arranged within the first container outer body, that defines a first interior space connected to a first opening of the first container and a second opening of the first container; a first container-side valve; and a plunger seal arranged within the first container barrel between the first opening, wherein the process comprises: aligning the first container and the second container; connecting the second container to the first container outer body, wherein the first container-side valve is configured to be activated, upon connection of the second container to the first container outer body, from a normally closed position that relatively reduces flow through the first opening of the first container into the first interior space to an activated position that relatively increases flow through the first opening of the first container into the first interior space; and applying a force to move the plunger seal within the first container barrel to change a volume of space within the first interior space between the first opening and the plunger seal.

In the thirteenth embodiment, the process further comprises: connecting a plunger to the plunger seal; and transferring the force from the plunger to the plunger seal to move the plunger seal within the first container barrel to change the volume of space within the first interior space between the first opening and the plunger seal.

In the thirteenth embodiment, the second container comprises a second container-side valve, and the process comprises: in connecting the second container to the first container outer body, bringing the first container-side valve into contact with the second container-side valve.

In the thirteenth embodiment, the process comprises: in bringing the first container-side valve into contact with the second container-side valve, activating, by the second container-side valve, the first container-side valve from the normally closed position to the activated position.

In the thirteenth embodiment, the second container comprises: a second container outer body that defines a second container barrel interior space connected a first opening of the second container, and the process comprises: activating the second container-side valve, upon connection of the second container to the first container outer body, from a normally closed position that relatively reduces flow through the first opening of the second container, from a normally closed position that relatively reduces flow through the first opening of the second container from the second container barrel interior space to an activated position that relatively increases flow through the first opening of the second container from the second container barrel interior space, to communicate the second container barrel interior space with the first interior space.

In the thirteenth embodiment, the second container-side valve comprises a valve stem, and the process comprises, upon connection of the second container to the first container outer body: bringing the valve stem into contact with the first container-side valve to activate the first container-side valve from the normally closed position to the activated position and the second container-side valve from the normally closed position to the activated position to communicate the second container barrel interior space with the first interior space through the valve stem.

In the thirteenth embodiment, the process further comprises: pre-loading the first interior space of the first container with a cell delivery solution.

In the thirteenth embodiment, the process further comprises: loading a mixture comprising a plurality of cells and a wash solution in the second container barrel interior space.

In the thirteenth embodiment, the process further comprises: after communicating the second container barrel interior space with the first interior space, applying a centrifugal force to the medical device system to move the plurality of cells from the second container barrel interior space through the second container-side valve in the activated position and the first container-side valve in the activated position into the first interior space.

In a fourteenth embodiment, an apparatus is described. The apparatus comprises: a first container for connecting to a second container, wherein the second container comprises: a second container outer body that defines a second container interior space; and a stem fluidly connected to the second container interior space, and wherein the first container comprises: a first container outer body, wherein the first container outer body defines a first interior space connected to a first opening of the first container; and a first container-side valve configured to, upon connection of the second container to the first container, be brought into contact with the stem to be activated from a normally closed position that relatively reduces flow through the first opening of the first container into the first interior space to an activated position that relatively increases flow through the first opening of the first container into the first interior space.

In the fourteenth embodiment, the first container further comprises a sheath defining a sheath interior space, and at least a portion of the first container outer body is configured to be removably inserted within the sheath interior space.

In the fourteenth embodiment, the first container-side valve is configured to be connected to a first end portion of the sheath, and the first container outer body is configured to be arranged within the sheath interior space to contact the first container-side valve such that upon activation of the first container-side valve from the normally closed position to the activated position the flow through the first opening of the first container into the first interior space is relatively increased.

In the fourteenth embodiment, the first container further comprises a sheath cap configured to be removably connected to a second end portion of the sheath with the first container outer body being enclosed within the sheath by the sheath cap.

In the fourteenth embodiment, the sheath cap comprises: a vent configured to vent air displaced from first interior space; and a filter arranged within the vent.

In the fourteenth embodiment, the first container further comprises a plunger seal arranged within the first interior space of the first container outer body, wherein the plunger seal is configured to receive a force to move the plunger seal within the first interior space to change a volume of space within the first interior space between the plunger seal and the first container-side valve.

In the fourteenth embodiment, the sheath cap defines a through hole through which gas in the interior space of the first container outer body is vented.

In the fourteenth embodiment, the plunger is configured to be removably connected to the plunger seal.

In the fourteenth embodiment, the first container further comprises a plunger seal arranged within the first interior space, wherein the plunger seal is configured to receive a force to move the plunger seal within the first interior space to change a volume of space within the first interior space between the plunger seal and the first container-side valve.

In the fourteenth embodiment, the apparatus further comprises a plunger configured to apply the force to the plunger seal to move the plunger seal within the first interior space to change the volume of space within the first interior space between the plunger seal and the first container-side valve.

In the fourteenth embodiment, the apparatus further comprises: a movable body arranged within the first interior space of the first container outer body, the movable body is configured to move within the first interior space between the plunger seal and the first container-side valve.

In the fourteenth embodiment, the movable body comprises a stainless-steel body.

In the fourteenth embodiment, the movable body is configured to be moved within the first interior space by a magnetic force exerted from outside of the first container.

In the fourteenth embodiment, the movable body defines one or more through holes connecting a first side of the movable body facing the first container-side valve and a second side of the movable body opposite to the first side of the movable body.

In the fourteenth embodiment, the first container further comprises a valve cap configured to be removably connected to the first container-side valve to prevent direct contact of the first container-side valve from an exterior of the valve cap.

In a fifteenth embodiment a process for preparing cell solution for delivery is disclosed. The process comprises receiving a formulated cell drug product (e.g., a suspension of cells in a cryoprotectant), separating the cells from the cryoprotectant with a cell wash solution, optionally, into a “cell delivery solution” to produce a cell solution for delivery to a subject/patient. In some variations, a “cell delivery solution” is comprised in a first container before cells are introduced into the first container. Addition of cells into a cell delivery solution results in preparation of a “cell solution for delivery,” a final cell preparation for implantation/delivery into a patient. It should be understood that the device disclosed here can be used for preparing solution that does not comprise cells (e.g., other macromolecules) for implantation into a patient.

In a sixteenth embodiment, a device that provides a means, e.g., an automated means, to wash a formulated cell drug product (e.g., a cell product containing cells and a cryopreserving agent) with a wash solution and deliver the cells into a cell delivery solution to prepare a cell solution for delivery is disclosed.

Further features as well as the structure and operation of various embodiments are described in detail below with reference to the accompanying drawings. In the drawings, like reference numbers indicate identical or functionally similar elements.

### BRIEF DESCRIPTION OF THE DRAWINGS

FIG. 1 shows a medical device system including a first container, a second container, a second container loading module and a dose loading module according to one embodiment.

FIG. 2 shows features of the first container according to one embodiment.

FIG. 3 shows features of the first container according to one modified embodiment.

FIG. 4 shows a process for assembling the first container according to one embodiment.

FIG. 5 shows features of the second container according to one embodiment.

FIG. 6 shows a disconnected arrangement and a connected arrangement of the first container and the second container according to one embodiment.

FIG. 7 shows a process for assembling the second container according to one embodiment.

FIG. 8 shows features of a second container loading module according to one embodiment.

FIG. 9 shows a process for preparing a cell solution for delivery according to one embodiment.

FIG. 10 shows a medical device system including a first container, a second container, a second container loading module and a dose loading module according to one embodiment.

FIG. 11 shows a disconnected arrangement and a connected arrangement of the first container and the second container according to one embodiment.

FIG. 12 shows a movable body used with the first container according to one embodiment.

FIGS. 13A-13D show examples of the movable body according to one embodiment.

FIG. 14 shows a first container mixing module according to one embodiment.

FIG. 15 shows a medical device system including a first container, a second container, a second container loading module and a dose loading module according to one embodiment.

FIG. 16 shows a disconnected arrangement and a connected arrangement of the first container and the second container according to one embodiment.

FIG. 17 shows the first container provided with a plunger head and a plunger seal according to one embodiment.

FIGS. 18 and 19 show a dispensing device applying a force to a plunger to advance the plunger seal according to one embodiment.

FIG. 20 shows a first container of a medical device system according to one embodiment

FIG. 21 shows a sheath and a first container-side valve according to one embodiment.

FIG. 22 shows a sheath cap according to one embodiment.

FIG. 23 shows a second container according to one embodiment.

FIG. 24 shows a medical device system including a first container and a second container according to one embodiment.

FIG. 25 shows different configuration of a first container during a preparation of a dose according to one embodiment.

FIG. 26 shows a medical device system according to one embodiment.

FIG. 27 shows a first container of a medical device system according to one embodiment.

FIG. 28 shows a dose tube of the first container of FIG. 26 according to one embodiment.

FIG. 29 shows an alternative dose tube retention mechanism according to one embodiment.

FIG. 30 shows a second container of the medical device system according to one embodiment.

FIG. 31 shows two connection positions between the first container and the second container.

FIG. 32 shows a sheath cap according to one embodiment.

FIG. 33 shows a loading adapter according to one embodiment.

FIG. 34 shows a delivery device guided by the loading adapter according to one embodiment.

### DETAILED DESCRIPTION

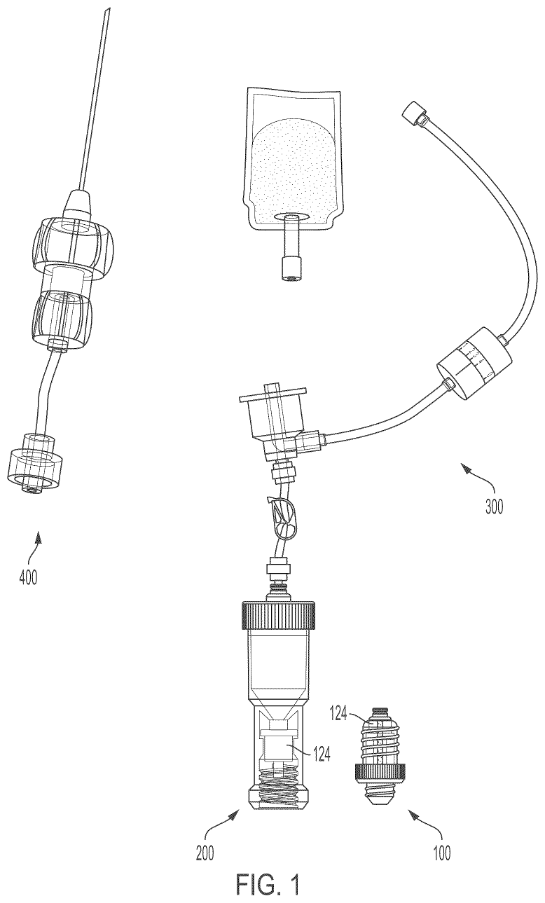

FIG. 1 illustrates an example of a medical device system 10 for implementing dose preparation according to one embodiment. The medical device system 10 may include a first container 100 (also referred to as a dose preparation tube), a second container 200 (also referred to as a wash tube) and a second container loading module 300 and a dose loading module 400.

### First Container (Dose Preparation Tube)

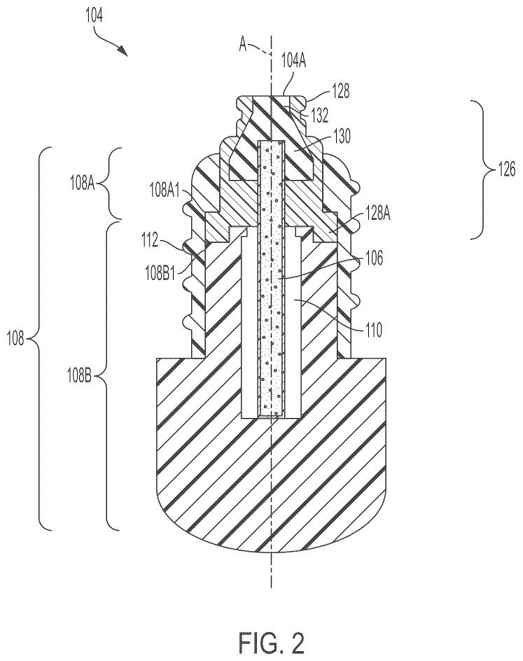

The first container 100, that is an apparatus, will now be described with reference to FIG. 2. The first container 100 may include a first tube body 104. The first tube body 104 may extend along a first tube body axis A between a first end of the first tube body 104 and a second end of the first tube body 104.

The first tube body 104 may define a first interior space 106. The first interior space 106 may be connected to a first opening 104A at the first end of the first tube body 104.

The volume of the first interior space 106 is not particularly limited. For example, the volume of the first interior space 106 may be selected based on a target dose volume of a dose to be prepared. As an example, the volume of the first interior space 106 may be selected to hold the target dose volume and to account for some additional dead volume (e.g., approximately 50 μl).

In some embodiments, the target dose volume may be at least about 1 microliter, at least about 2 microliters, at least about 3 microliters, at least about 4 microliters, at least about 5 microliters, at least about 6 microliters, at least about 7 microliters, at least about 7.2 microliters, at least about 7.4 microliters, at least about 7.6 microliters, at least about 7.8 microliters, at least about 8 microliters, at least about 8.1 microliters, at least about 8.2 microliters, at least about 8.3 microliters, at least about 8.4 microliters, at least about 8.5 microliters, at least about 8.6 microliters, at least about 8.7 microliters, at least about 8.8 microliters, at least about 8.9 microliters, at least about 9 microliters, at least about 9.5 microliters, at least about 10 microliters, at least about 11 microliters, at least about 12 microliters, at least about 13 microliters, at least about 14 microliters, at least about 15 microliters, at least about 20 microliters, at least about 30 microliters, at least about 100 microliters, at least about 1 mL, at least about 10 mL, at least about 100 mL, at least about 500 mL, or at least about 800 mL.

In some embodiments, the target dose volume may be less than or equal to about 1000 mL, less than or equal to about 800 mL, less than or equal to about 500 mL, less than or equal to about 100 mL, less than or equal to about 10 mL, less than or equal to about 1 mL, less than or equal to about 100 microliters, or less than or equal to about 90 microliters, or less than or equal to about 80 microliters, or less than or equal to about 70 microliters, or less than or equal to about 60 microliters, or less than or equal to about 50 microliters, or less than or equal to about 40 microliters, or less than or equal to about 30 microliters, or less than or equal to about 20 microliters, or less than or equal to about 15 microliters, or less than or equal to about 12 microliters, or less than or equal to about 10 microliters, or less than or equal to about 9.9 microliters, or less than or equal to about 9.8 microliters, or less than or equal to about 9.7 microliters, or less than or equal to about 9.6 microliters, or less than or equal to about 9.5 microliters, or less than or equal to about 9.4 microliters, or less than or equal to about 9.3 or less than or equal to about 9.2 microliters, or less than or equal to about 9.1 microliters, or less than or equal to about 9 microliters, or less than or equal to about 8.8 microliters, or less than or equal to about 8.2 microliters, or less than or equal to about 8 microliters, or less than or equal to about 7 microliters, or less than or equal to about 6 microliters, or less than or equal to about 5 microliters.

In some embodiments, the target dose volume may be approximately 150 microliters, approximately 1.8 microliters or approximately 2.0 microliters.

Combinations of the above referenced ranges are also possible. For example, in some embodiments, the target dose volume is about 100 microliters to about 1mL, or 1 mL to about 1000 mL, or about 10 mL to about 800 mL, or about 100 mL to about 500 mL, or about 1 microliter to about 100 microliters, or about 2 microliters to about 60 microliters, or about 3 microliters to about 30 microliters, or about 4 microliters to about 20 microliters, or about 5 microliters to about 18 microliters or about 6 microliters to about 16 microliters or about 7 microliters to about 14 microliters or about 8 microliters to about 10 microliters or about 8.5 microliters to about 9.5 microliters or about 8.9 microliters to about 9.1 microliters, or about 9 microliters to about 9.1 microliters.

As an example, the first tube body 104 may include a first container outer body 108 and a first container barrel 110 arranged within the first container outer body 108.

A material or materials for forming the first container outer body 108 is not particularly limited. Examples of the material or materials for forming the first container outer body 108 may include low leachable and/or extractable materials such as cyclic olefin polymer, cyclic olefin copolymer, perfluoroalkoxy alkane, ethylene tetra fluoroethylene, cyclic block copolymers, polyethylene, polypropylene and polycarbonates. The material or materials for forming the first container may also be selected based on contemplated methods of manufacture, such as injection molding.

The first container outer body 108 may have a geometry or structure for directly or indirectly connecting the first container 100 to the second container 200. A surface of the first container outer body 108 may include a first part 112 of a connection or coupling mechanism to directly or indirectly connect the first container 100 to a second container. The connection or coupling mechanism is not particularly limited. As an example, the first part 112 of the connection or coupling mechanism may include one part of a threaded connection, while the second container 200 may include another part of the threaded connection that detachably connects to the one part of the threaded connection to connect the second container 200 to the first container 100. The one part of the threaded connection can be a one of a male thread (as shown in FIG. 2) and a female thread, while the another part of the threaded connection provided to the second container 200 may be the other of the male thread and the female thread.

The first container outer body 108 may have a geometry or structure that allows the first container 100, when attached to the second container 200, to be fitted within a compartment of a centrifuge as described in more detail below.

The first container barrel 110 may be arranged within the first container outer body 108 along the first tube body axis A. The first container barrel 110 may define the first interior space 106. The first container barrel 110 may be open-ended on a side closer to the first end of the first tube body 104 to connect the first interior space 106 to the first opening 104A of the first tube body 104. The first container barrel 110 may be close-ended on a side closer to the second end of the first tube body 104.

The dimensions of the first container barrel 110 is not particularly limited. For example, a length of the first container barrel 110 along the first tube body axis A and an inner diameter of the first container barrel 110 in a direction perpendicular to the first tube body axis A may be selected based on the target dose volume of the dose to be prepared.

The first container barrel 110 may be formed as a unitary structure or an assembly of a plurality of structures. A material or materials for forming the first container barrel 110 is not particularly limited. The material or materials for forming at least an interior surface and any other cell contacting surface of the first container barrel 110 may be a biocompatible material or materials such as a biocompatible polymer. The material or materials for forming the first container barrel 110 may be selected to minimize cell adhesion to the interior surface and any other cell contacting surface of the first container barrel 110. Examples of the materials or materials for forming the first container barrel 110 may include resins such as polypropylene and polycarbonate.

Referring to FIG. 1, the medical device system 10 may include a first flow path control module 124 for controlling a flow between the second container 200 and the first container 100.

Referring to FIGS. 1 and 2, the first container 100 may include a first portion of the first flow path control module 124. The first portion of the first flow path control module 124 is configured to be activated from a normally closed position to an activated position, and to be deactivated from the activated position to the normally closed position. In the normally closed position, a flow into and out of the first interior space 106 through the first opening 104A is reduced relative to the activation position or completely restricted. In the activated position, a flow into and out of the first interior space 106 through the first opening 104A is increased relative to the normally closed position.

The structure and geometry of the first portion of the first flow path control module 124 is not particularly limited. One example of the first portion of the first flow path control module 124 is described in detail below.

The first flow path control module 124 may include a valve system. The first portion of the first flow path control module 124 may include a first container-side valve 126. The first container-side valve 126 may be provided to the first end of the first tube body 104. The first container-side valve 126 may include a first valve casing 128 attached to the first container outer body 108. Although the first container-side valve 126 is described as a separate structure from other parts of the first container 100 such as the first container outer body 108, portions of the first container-side valve 126 such as the first valve casing 128 may be formed as a unitary structure with the first container outer body 108.

The outer surface of the first valve casing 128 may have a geometry or structure for directly or indirectly connecting the first container-side valve 126 of the first container 100 to a third container. The outer surface of the first valve casing 128 may include a first part of a connection or coupling mechanism for directly or indirectly connecting the first container 100 to the third container. An example of the first part of the connection or coupling mechanism may include one part of a threaded connection, while the third container may include another part of the threaded connection that detachably connects the third container to the first container-side valve 126 of the first container 100. The third container may include a medical device such as a syringe for receiving a dose of a cell solution for delivery from the first container 100 and delivering the dose to a subject. The third container may include a connector to the medical device through which the dose can be moved from the first container 100 to the medical device such as the syringe.

An interior surface of the first valve casing 128 may define a first valve casing interior space 130 leading to the first opening 104A of the first tube body 104. The first container-side valve 126 may include a first seal 132 arranged within the first valve casing interior space 130 of the first valve casing 128. The first seal 132 is configured to provide the normally closed position and the activated position of the first container-side valve 126. The structure of the first seal 132 is not particularly limited. An example of the first seal 132 is a self-sealing seal.

The first seal 132 may be arranged in the first valve casing interior space to control a flow through the open end of the first container barrel 110 into the first interior space 106. For example, the open end of the first container barrel 110 may extend along the first tube body axis A into the first valve casing interior space 130 to abut the first seal 132.

The first seal 132 may have a normally closed position that relatively reduces or completely restricts flow through the first seal 132. The normally closed position of the first seal 132 may be a sealed position of the self-sealing seal.

The first seal 132 may be activated from the normally closed position to an activated position. The activated position of the first seal 132 allows for relatively increased flow through the first seal 132 and the open end of the first container barrel 110 into the first interior space 106 of the first container barrel 110 (and the first tube body 104). For example, the first seal 132 may be activated from the normally closed position to the activated position by deforming the self-sealing seal to provide a pathway for flow through the first seal 132. The first seal 132 may be activated by a valve stem to be described below.

The first seal 132 may be made of a resilient material such as rubber, silicone rubber, a flexible plastic or other resilient polymer.

Referring to FIG. 2, the first container outer body 108 may have a geometry or structure for connecting the first portion of a first flow path control module 124 to the first container outer body 108 will be described.

To aid in the assembly of the first container 100, the first container outer body 108 may be provided as a plurality of parts. For example, the first container outer body 108 may include a first outer body part 108A and a second outer body part 108B. The first outer body part 108A and the second outer body part 108B may be arranged sequentially along the first tube body axis A such that the first outer body part 108A is arranged closer to the first end of the first tube body 104 and the second outer body part 108B is arranged closer to the second end of the first tube body 104 along the first tube body axis A.

The second outer body part 108B may define an interior space extending along the first tube body axis A. For example, the second outer body part 108B may have a tubular inner surface that defines the interior space of the second outer body part 108B. The interior space of the second outer body part 108B may be dimensioned to allow the first container barrel 110 to be arranged therein. The first container barrel 110 may be connected to the tubular inner surface of the second outer body part 108B by, for example, press fitting the first container barrel 110 within the tubular inner surface of the second outer body part 108B and/or, for example, adhering the cylindrical outer surface of the first container barrel 110 to the tubular inner surface of the second outer body part 108B.

The first outer body part 108A and the second outer body part 108B may have geometries or structures that cooperate to connect the first container outer body 108 to the first container-side valve 126.

The first outer body part 108A may define an interior space extending along the first tube body axis A. For example, the first outer body part 108A may have a tubular inner surface that defines the interior space of the second outer body part 108B. The first valve casing 128 may have a cylindrical outer surface having an outer diameter that matches an inner diameter of the tubular inner surface of the first outer body part 108A such that the first valve casing 128 is coaxially arranged within the interior space defined by the tubular inner surface of the first outer body part 108A. The first valve casing 128 may be connected to the tubular inner surface of the first outer body part 108A by, for example, press fitting the first valve casing 128 within the tubular inner surface of the first outer body part 108A and/or, for example, adhering the cylindrical outer surface of the first valve casing 128 to the tubular inner surface of the first outer body part 108A.

An outer surface of the first valve casing 128 may include a first valve casing flange 128A. The first outer body part 108A may include a first sandwiching surface 108A1, and the second outer body part 108B may include a second sandwiching surface 108B1. Further, the first sandwiching surface 108A1 and the second sandwiching surface 108B1 may be separated by a gap in which the first valve casing flange 128A may be arranged to be sandwiched by the first sandwiching surface 108A1 and the second sandwiching surface 108B1. Still further, the first outer body part 108A and the second outer body part 108B may have corresponding surfaces that can be connected by, for example, press fitting and/or adhesive while the first valve casing flange 128A is sandwiched by the first sandwiching surface 108A1 and the second sandwiching surface 108B1 to thereby connect the first container outer body 108 to the first container-side valve 126. In this connected state, when the first seal 132 is activated from the normally closed position to the activated position, flow from the second container 200 into the first interior space 106 within the second outer body part 108B, or flow from the first interior space 106 within the second outer body part 108B into the third container may occur.

Although the first container 100 is described above as including a plurality of separate parts such as the first outer body part 108A, the second outer body part 108B, the first valve casing 128 and the first container barrel 110, two or more of the plurality of separate parts may be provided as a single structure.

The dimensions of the first container 100 is not particularly limited. For example, a length of the first container 100 along the first tube body axis A and an outer diameter of the first container 100 in a direction perpendicular to the first tube body axis A can be selected to allow the first container 100, together with the second container, to be fitted within the compartment of the centrifuge, as described below.

### First Container (Dose Preparation Tube)—Modification

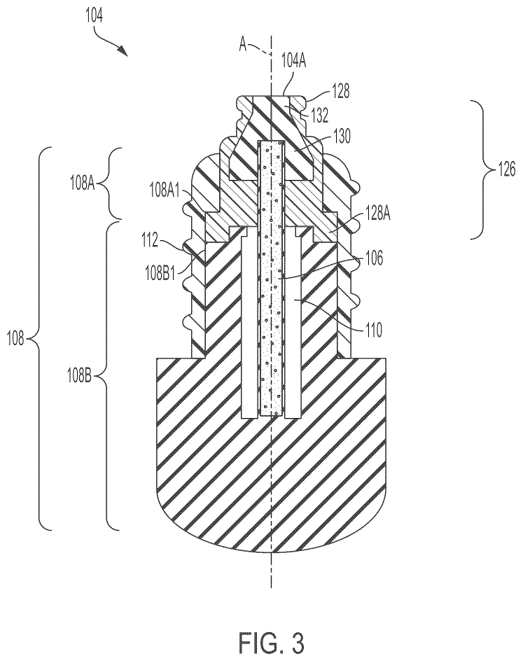

A modified embodiment of the first container 100 will now be described with reference to FIG. 3. The modified embodiment of the first container 100 is similar to the first container 100 described above. In the modified embodiment, a portion of the second outer body part 108B of the first container outer body 108 and a portion of the first container barrel 110, as described above, may be replaced with a malleable material. The malleable material may be selected to be squeezed to decrease the volume of the first interior space. As an example, the malleable material may include fluorinated ethylene propylene, a polyimide material such as Kapton®, polyvinyl chloride, polypropylene, silicone, polyurethane or any suitable material.

### Process for Assembling First Container (Dose Preparation Tube)

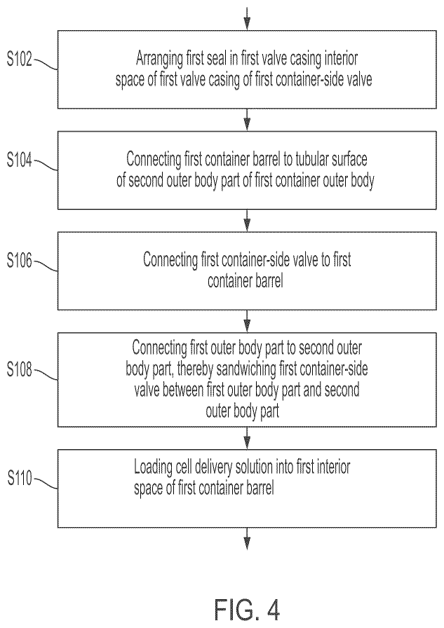

Next, a process for assembling the first container 100 will be described with reference to FIG. 4.

The process for assembling the first container 100 may include a step S102 of arranging first seal 132 in the first valve casing interior space 130 of the first valve casing 128 of the first container-side valve 126.

The process for assembling the first container 100 may include a step S104 of connecting the first container barrel 110 to the tubular surface of the second outer body part 108B by, for example, press fitting the first container barrel 110 within the tubular inner surface of the second outer body part 108B and/or for example, adhering the cylindrical outer surface of the first container barrel 110 to the tubular inner surface of the second outer body part 108B.

The process for assembling the first container 100 may include a step S106 of connecting the first container-side valve 126 to the first container barrel 110. The step S106 may include arranging the first seal 132 to the first container barrel 110 such that when the first seal 132 is activated from the normally closed position to the activated position, the activated position of the first seal 132 allows for relatively increased flow through the first seal 132 and the open end of the first container barrel 110 into the first interior space 106 of the first container barrel 110.

The first container-side valve 126 may be connected to the first container barrel 110 by connecting the first valve casing flange 128A of the first valve casing 128 to the second sandwiching surface 108B1 of the second outer body part 108B.

The process for assembling the first container 100 may include a step S108 of connecting the first outer body part 108A to the second outer body part 108B. For example, the corresponding surface of the first outer body part 108A and the second outer body part 108B may be connected by, for example, press fitting and/or adhesive. In connecting the first outer body part 108A to the second outer body part 108B, the first valve casing flange 128A may be sandwiched between the first sandwiching surface 108A1 and the second sandwiching surface 108B1.

The process for assembling the first container 100 may include a step S110 of loading a predetermined volume of cell delivery solution into the first interior space 106 of the first container barrel 110. The cell delivery solution may be loaded into the first interior space 106 through the first container-side valve 126 that is activated from the normally closed position to the activated position. The predetermined volume of the cell delivery solution to be loaded into the first interior space 106 may be selected based on target dose volume to be prepared. For example, the predetermined volume of the cell delivery solution may be selected to be the same as the volume of the first interior space 106. By selecting the predetermined volume of the cell delivery solution to be the same as the volume of the first interior space 106, any additional loading of cells from the second container 200 into the first interior space 106 may displace the same or substantially the same volume of the cell delivery solution such that the first interior space 106 having the cells and the cell delivery solution (together to be considered a prepared dose) will occupy the entirety or substantially the entirety of the first interior space 106. In another example, the predetermined volume of the cell delivery solution may be selected such that the predetermined volume of the cell delivery solution with a predetermined volume of the cells loaded from the second container 200 into the first interior space 106 will occupy the entirety or substantially the entirety of the first interior space 106.

As used herein, the cell delivery solution may be any solution that is added to unwashed cells (directly on thawed cells) or washed cells so that the cells can be administered to a subject. The cell delivery solution can contain no, minimal or trace amounts of cryoprotectant and/or cell wash solution, which the cells were stored and/or washed in prior to contact with the cell delivery solution, that were not fully removed from the container after a supernatant discard process, which can occur after an optional centrifuge step of the container, which can optionally form a cell pellet or concentrated cell solution. This supernatant discard process reduces the concentration and/or removes components of a stored sample that are not desired for delivery to a subject, such as the cryoprotectant. The cell delivery solution can be used to reconstitute a cell solution for delivery following thawing of cells prior to administration for clinical use.

As used herein, cell wash solution is any solution that is added to a container containing cells and a cryoprotectant after removal of that container from a below 0° C. environment. As used herein, the term “cryoprotectant” refers to a substance that is used to reduce or eliminate cell damage caused by freezing and thawing processes which are inevitably accompanied by ice crystal formation and ionic and osmotic imbalance when cells and/or tissues are preserved at temperatures below 0° C. The cryoprotectant is not limited to a certain substance, as long as it is able to reduce cell damage during below 0° C. preservation. Examples thereof may include a permeating-type cryoprotectant such as dimethyl sulfoxide (DMSO), glycerol, propylene glycol, ethylene glycol, etc., or a non-permeating-type cryoprotectant such as sucrose, carboxymethylcellulose salts, carboxymethylcellulose (CMC), monosaccharide, disaccharide, etc., but are not limited thereto.

The process for assembling the first container 100 is described as including steps S102 to S110 performed in the described order. However, the steps S102 to S110 may be performed in different orders.

The above-described process for assembling the first container 100 may occur in an aseptic environment (e.g., a biosafety cabinet) or near aseptic environment to eliminate or minimize contamination of the first interior space 106 after the first container 100 is fully assembled.

### Second Container (Wash Tube)

Returning to FIG. 1, the medical device system 10 may include the second container 200 (also referred to as a wash tube).

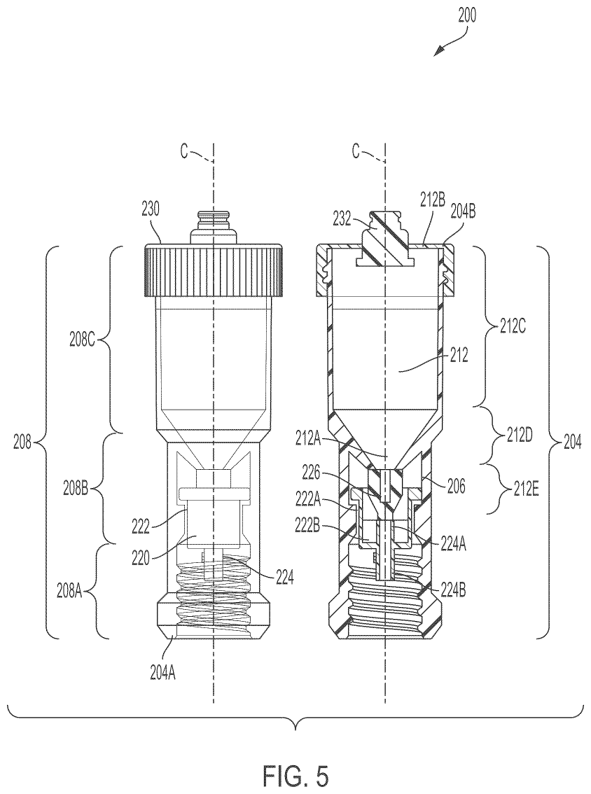

Referring to FIG. 5, the second container 200, that is an apparatus, may include a second tube body 204. The second tube body 204 may extend along a second tube body axis C between a first end 204A of the second tube body 204 and a second end 204B of the second tube body 204.

The dimensions of the second container 200 is not particularly limited. For example, a length of the second container 200 along the second tube body axis C and an outer diameter of the second container 200 in a direction perpendicular to the second tube body axis C can be selected to allow the second container 200 with the first container 100 connected thereto be fitted within the compartment of the centrifuge.

The second tube body 204 may include a second container outer body 208. The second container outer body 208 may have a geometry or structure that allows the second container 200, when connected to the first container 100, to be fitted within a compartment of a centrifuge as described in more detail below.

A material or materials for forming the first and/or second container outer body 208 is not particularly limited. Examples of the material or materials for forming the second container outer body 208 may include low leachable and/or extractable materials such as cyclic olefin polymer, cyclic olefin copolymer, perfluoroalkoxy alkane, ethylene tetra fluoroethylene, cyclic block copolymers, polyethylene, silicone, polypropylene and polycarbonates. The material or materials for forming the first and/or second container may also be selected based on contemplated methods of manufacture, such as injection molding.

As contemplated herein, any component, and in particular any component of the first container and the second container, that would be in contact with a solution may be of a low leachable and/or extractable material.

Referring to FIG. 5, the second container outer body 208 may include a first section 208A, a second section 208B and a third section 208C arranged in the recited order from the first end 204A of the second tube body 204 to the second end 204B of the second tube body 204. The first section 208A and the second section 208B may be formed as a single structure. The first section 208A and the second section 208B may define a second interior space 206 between a first opening (that is also the first end 204A) and a second opening. Further, the third section 208C may be a separate structure connected to the second section 208B. The third section 208C may be connected to the second section 208B such that a portion of the third section 208C is arranged in the second interior space 206.

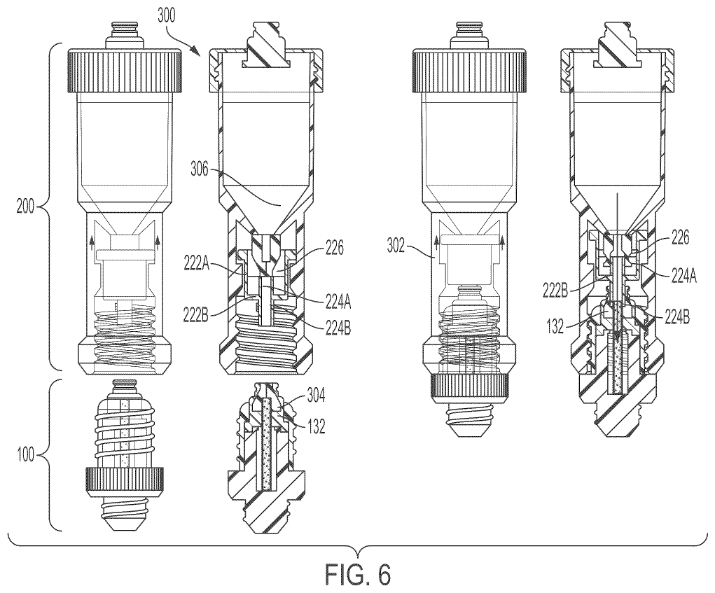

The first section 208A of the second container outer body 208 may have a geometry or structure for directly or indirectly connecting the second container 200 to the first container 100, as described above. A surface of the first section 208A of the second container outer body 208 may include a second part of the connection or coupling mechanism to directly or indirectly connect the second container 200 to the first container 100. As an example, an inner surface of the first section 208A of the second container outer body 208 may include a female thread (as shown in FIGS. 5 and 6) configured to be connected to the male thread (as shown in FIGS. 2 and 6) of the first part 112 of the first container outer body 108.

Referring to FIG. 5, the third section 208C of the second container outer body 208 may define a second container barrel interior space 212. The third section 208C may extend along the second tube body axis C between a first end of the third section 208C and a second end of the third section 208C. The third section 208C may define a first opening 212A along the second tube body axis C between the first end 204A of the second tube body 204 and the second end 204B of the second tube body 204. The third section 208C may define a second opening 212B along the second tube body axis C closer to the second end 204B of the second tube body 204 than the first opening 210A of the second container barrel 210.

The volume of the second container barrel interior space 212 is not particularly limited. For example, the volume of the second container barrel interior space 212 may be selected based on the target dose volume of the dose to be prepared in the first container 100 and a volume of therapeutic substance in wash buffer to be loaded in the second container barrel interior space 212. In some embodiments, the therapeutic substance to be delivered are cells, or other particles having a certain diameter. It should be appreciated, however, that the therapeutic substance is not limited to cells or particles. Wherever discussed hereinafter, “cells” may be substituted with any other therapeutic substance, as appropriate.

In some embodiments, the cells are pluripotent stem cell (PSC)-derived cells, progenitor or precursor cells thereof. PSCs can be embryonic stem cells or induced pluripotent stem cells. In some embodiments, the cells are PSCs. In some embodiments, the cells are dopaminergic neuron cells, progenitor or precursor cells thereof, and, in some embodiments, may be iPSC-derived dopaminergic neuron cells, progenitor or precursor cells thereof. It should be appreciated however, the types of cells are not particularly limited. Other type of cells, such as mesenchymal stem cells, hematopoietic stem cells, embryonic stem cells or induced pluripotent stem cells, red blood cells, platelets, chondrocytes, skin cells, immune cells (e.g. tumor infiltrating lymphocytes, viral reconstitution T cells, dendritic cells, regulator T cells, macrophages), neural crest stem cells, neurons, glia, smooth muscle, cardiac tissue, chondrocytes, osteocytes, glial restricted progenitors, astrocytes, oligodendrocytes, neuroblast cells, megakaryoblasts, megakaryocytes, monoblasts, monocytes, macrophages, myeloid dendritic cells, proerythroblasts, erythroblasts, normoblasts, reticulocytes, thrombocytes, myeloblasts, progranulocytes, neutrophilic myelocytes, neutrophilic band cells, neutrophils, eosinophilic myelocytes, eosinophilic band cells, eosinophils, basophilic myelocytes, basophilic band cells, basophils, committed lymphoid progenitors, pre-NK cells, NK lymphoblasts, NK cells, thymocytes, T-lymphoblasts, T-cells, plasmacytoid dendritic cells, pre-B cells, B-lymphoblasts, B cells, plasma cells, osteoblasts, chondrocytes, myoblasts, myotubes, fibroblasts, adipocytes, mesoderm, ectoderms, primordial germ cells, sperm, eggs, definitive endoderm, myeloid progenitor cells, microglial cells, myeloid cells, cardiac cells (e.g., cardiomyocyte cells such as those of the atrium or ventricle), progenitor or precursor cells thereof, or any other suitable type of cell may be processed.

The third section 208C may have a geometry or structure for directing a displacement of the cells in the wash buffer by centrifugal force applied by the centrifuge towards the first opening 212A of the third section 208C. The geometry or structure of the third section 208C is not particularly limited. For example, the third section 208C may include a first part 212C, a second part 212D and a third part 212E arranged in the recited order along the second tube body axis C from the second opening 212B of the third section 208C and the first opening 212A of the third section 208C. The first part 212C may have a cylindrical shape having a first diameter. The second part 212D can be a conical shape having a varying diameter that reduces along the second tube body axis C in a direction from the second opening 212B towards the first opening 212A from the first diameter to a second diameter. The third part 212E may be a cylindrical shape having the second diameter.

Referring to FIG. 5, the second container 200 may include a cover 230 configured to be connected to the third section 208C to cover the second opening 212B of the third section 208C. The cover 230 and the second tube body 204 each have portions of a connection or coupling mechanism to directly or indirectly connect the cover 230 to the second tube body 204. As an example, the third section 208C may include one part of a threaded connection, while the cover 230 may include another part of the threaded connection that detachably connects the cover 230 to the second container outer body 208. The one part of the threaded connection can be one of a male thread (as shown in the drawings) and a female thread, while the another part of the threaded connection provided to the cover 230 may be the other of the male thread and the female thread (as shown in the drawings).

Referring to FIG. 5., the second container 200 may include a second flow path control module 232 arranged to the cover 230. The second flow path control module 232 may include a luer valve that may be activated from a normally closed position to an activated position to permit flow into the second container barrel interior space 212, and to be deactivated from the activated position to the normally closed position to reduce to eliminate flow into the second container barrel interior space 212. The number of second flow path control modules 232 may be numerous. For example, there may be two, three, or more second flow path control modules 232. In some embodiments, one second flow path control module may be used to introduce a cell wash solution to the second container, which another second flow path control module may be used to vent air.

Referring to FIG. 5, the second container 200 may include a second portion of the first flow path control module 124. The second portion of the first flow path control module 124 is configured to be activated from a normally closed position to an activated position, and to be deactivated from the activated position to the normally closed position. In the normally closed position, a flow out of the second container barrel 210 through the first opening 212A of the third section 208C is relatively reduced or completely restricted. In the activated position, a flow out of the third section 208C through the first opening 212A is increased relative to the normally closed position.

As described above, the first flow path control module 124 may include the valve system. The second portion of the first flow path control module 124 may include a second container-side valve 220. The second container-side valve 220 may be provided in the second section 208B of the second container outer body 208, as described below.

The second container-side valve 220 may include a valve cap 222, a valve stem 224 and a second seal 226.

The valve cap 222 may include a cylinder 222A and an attachment surface 222B. The cylinder 222A may be arranged within the second section 208B of the second container outer body 208. An inner surface of the second section 208B of the second container outer body 208 is sized to limit movement of the cylinder 222A to movement along a predetermined range of the second tube body axis C in the direction from the first end 204A of the second tube body 204 toward the second end 204B of the second tube body 204.

The valve stem 224 may be connected, at an intermediate portion of the valve stem 224 along the length of the valve stem 224, to the attachment surface 222B. A first portion 224A of the valve stem 224 on a side of the attachment surface 222B facing the third section 208C of the second container outer body 208 is exposed while being surrounded radially by the cylinder 222A of the valve cap 222, and a second portion 224B of the valve stem 224 on a side of the attachment surface 222B facing the first end 204A is also exposed. The valve stem 224 is connected by the attachment surface 222B to the cylinder 222A such that when the cylinder 222A moves along the predetermined range of the second tube axis C, the valve stem 224 also moves along the second tube axis C. A portion of the valve stem 224 including a first end of the valve stem 224 may have a tapered geometry that reduces an outer diameter of the portion of the valve stem 224 in a direction approaching the first end of the valve stem 224. A portion of the valve stem 224 including a second end of the valve stem 224 may have a tapered geometry that reduces an outer diameter of the portion of the valve stem 224 in a direction approaching the second end of the valve stem 224. The tapered geometries of the portions of the valve stem 224 including the first end and the second end of the valve stem 224 may improve the ability of the valve stem 224 to activate the first seal 132 and the second seal 226 as described in detail below.

The second seal 226 may be arranged to be connected to the third part 212E of the third section 208C of the second container outer body 208. The second seal 226 is configured to provide a normally closed position and an activated position of the second container-side valve 220. An example of the second seal 226 is a self-sealing seal.

The second seal 226 has the normally closed position that relatively reduces or completely restricts flow through the second seal 226. The second seal 226 may be activated from the normally closed position to the activated position. The activated position of the second seal 226 allows for relatively increased flow through the second seal 226. For example, the second seal 226 may be activated from the normally closed position to the activated position by deforming the self-sealing seal to provide a pathway for flow through the second seal 226. The second seal 226 may be activated by the valve stem 224 as described below.

Referring to FIG. 6, activation of the first container-side valve 126 and the second container-side valve 220 by the valve stem 224 through connection of the first container 100 and the second container 200 will be described.

As described above, the first container outer body 108 may include the male thread, and the first section 208A of the second container outer body 208 may include the female thread. The first container 100 and the second container 200 may be connected by aligning the first tube body axis A and the second tube body axis C, and engaging the male thread of the first container outer body 108 with the female thread of the first section 208A of the second container outer body 208 through rotation of the first container 100 relative to the second container 200 to advance the first container-side valve 126 towards the second container-side valve 220. As the first container-side valve 126 continues to advance towards the second container-side valve 220, the tapered portion of the valve stem 224 closest to the first seal 132 advances past the first valve casing 128 to contact and activate the first seal 132 from the normally closed position to the activated position.

As the male thread of the first container outer body 108 continues to engage the female thread of the first section 208A to advance the first container-side valve 126 towards the second container-side valve 220, the first valve casing 128 of the first container-side valve 126 may be brought into contact with the valve cap 222. For example, the first valve casing 128 may be brought into contact with the attachment surface 222B of the valve cap 222. A force in a direction of the first container 100 towards the second container 200 resulting from the advancement of the first container-side valve 126 towards the second container-side valve 220 may be transferred from by the first valve casing 128 to the valve cap 222 to move the valve cap 222 having the valve stem 224 attached thereto along the predetermined range of the second tube body axis C in the direction of the second end 204B of the second tube body 204 towards the second seal 226. As the valve cap 222 is moved towards the second seal 226, the tapered portion of the first portion 224A of the valve stem 224 may contact and activate the second seal 226 from the normally closed position to the activated position. In a state where the valve cap 222 is moved to an end of the predetermined range closest to the second end 204B of the second tube body 204, the tapered portions of the valve stem 224 contact the first seal 132 and the second seal 226, respectively, to activate the first container-side valve 126 and the second container-side valve 220 to form a pathway for flow from the second interior space 206 of the second tube body 204 to the first interior space 106 of the first tube body 104.

Referring to FIG. 6, deactivation of the first container-side valve 126 and the second container-side valve 220 through disconnection of the first container 100 the second container 200 will be described.

The first container 100 and the second container 200 may be disconnected by disengaging the male thread of the first container outer body 108 from the female thread of the first section 208A of the second container outer body 208 through counter-rotation of the first container 100 relative to the second container 200 to retract the first container-side valve 126 away from the second container-side valve 220. As the first container-side valve 126 continues to retract from the second container-side valve 220, the tapered portion of the second portion 224B of the valve stem 224 that activated the first container-side valve 126 is retracted away from the first seal 132 to deactivate the first seal 132 from the activated position to the normally closed position to thereby reduce or completely eliminate flow through the first seal 132 of the first container-side valve 126.

### Process for Assembling Second Container (Wash Tube)

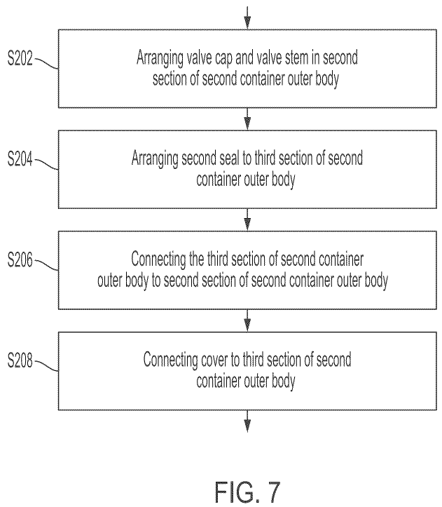

Next, a process for assembling the second container 200 will be described with reference to FIG. 7.

The process for assembling the second container 200 may include a step S202 of arranging the valve cap 222 and the valve stem 224 connected to the valve cap 222 in the second section 208B of the second container outer body 208. The valve cap 222 and the valve stem 224 may be inserted through the second opening of the single structure including the first section 208A and the second section 208B to be arranged in the second interior space 206. The valve cap 222 and the valve stem 224 may be arranged in the second interior space 206 such that the first portion 224A of the valve stem 224 faces the third section 208C of the second container outer body 208 and such that the second portion 224B of the valve stem 224 faces the first opening 104A of the second tube body 204.

The process for assembling the second container 200 may include a step S204 of arranging the second seal 226 to the third part 212E of the third section 208C of the second container outer body 208.

The process for assembling the second container 200 may include a step S206 of connecting the third section 208C of the second container outer body 208 to the second section 208B such that a portion of the third section 208C is arranged in the second interior space 206. For example, the third section 208C may be connected to the second section 208B such that the third part 212E (having the second seal 226 arranged to the third part 212E) and a portion of the second part 212D of the third section 208C are arranged in the second interior space 206.

The third section 208C may be connected to the second section 208B to define the predetermined range along which the cylinder 222A of the second container-side valve 220 moves. Further, second container-side valve 220 is in the normally closed position at the position at which the third section 208C is connected to the second section 208B.

The process for assembling the second container 200 may include a step S208 of connecting the cover 230 having the second flow path control module 232 arranged to the cover 230 to the third section 208C of the second container outer body 208 to cover the second opening 212B of the second container barrel interior space 212.

The above-described process for assembling the second container 200 may occur in an aseptic environment (e.g., a biosafety cabinet) to minimize or eliminate contamination of the second container barrel interior space 212 and the second interior space 206 after the second container 200 is fully assembled.

### Second Container Loading Module

Referring to FIG. 1, the medical device system 10 may include a second container loading module 300 for loading a plurality of cells and a cell wash solution into the second container barrel interior space 212 of the second container 200.

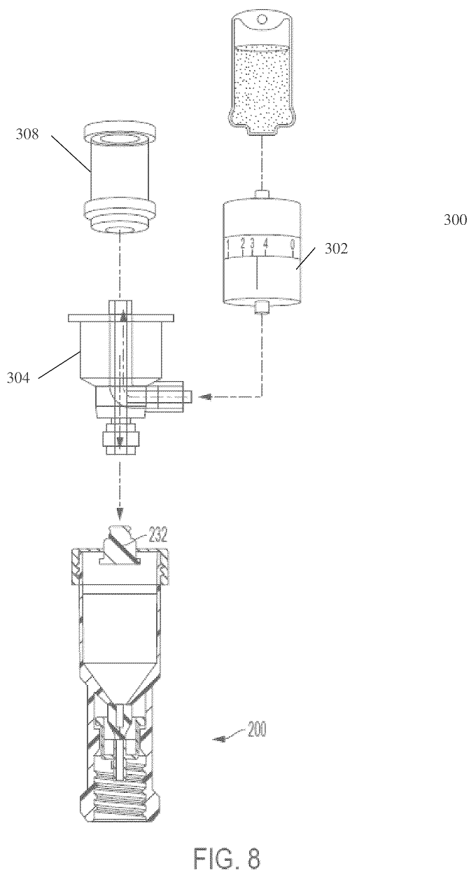

The second container loading module 300 will be described with reference to FIG. 8. The second container loading module 300 may include a flow regulator 302, an adapter 304 and a cell vial 308. The cell vial 308 may hold a mixture of a predetermined number of cells and cryoprotectant. The flow regulator 302 may fluidly connect a source of wash solution to the adapter 304. The adapter 304 may fluidly connect the flow regulator 302 to the cell vial 308. The adapter 304 may separately fluidly connect the cell vial 308 to the second container 200.

The flow regulator 302 may regulate the flow of wash solution to the cell vial 308 via the adapter 304 to, for example, 1 drop (approximately 50 microliters) per second to prevent osmotic shock to the cells contained in the cell vial 308.

The adapter 304 may fluidly direct the wash solution, the flow of which is regulated by the flow regulator 302, to the cell vial 308. Further, the adapter 304 may be directly or indirectly connected to the second flow path control module 232 to fluidly direct a mixture of the cells, the cryoprotectant and the wash solution from the cell vial 308 through the second flow path control module 232 into the second container barrel interior space 212 of the third section 208C of the second container outer body 208.

As an alternative to the second container loading module 300, a solution of cells may be placed directly in the second container barrel interior space 212 of the second container 200, and the wash solution may be fed into the second container battel interior space 212 of the second container 200 before or after the solution of cells is placed in the second container barrel interior space 212 of the second container 200. The mixture of the solution of cells and wash solution in the second container 200 is then centrifuged to transfer a portion of the cells into the first container 100 as described below.

As yet another alternative, a solution of cells can be placed in the first container 100 directly, and the first container 100 is then centrifuged. Process for Preparing a cell solution for delivery

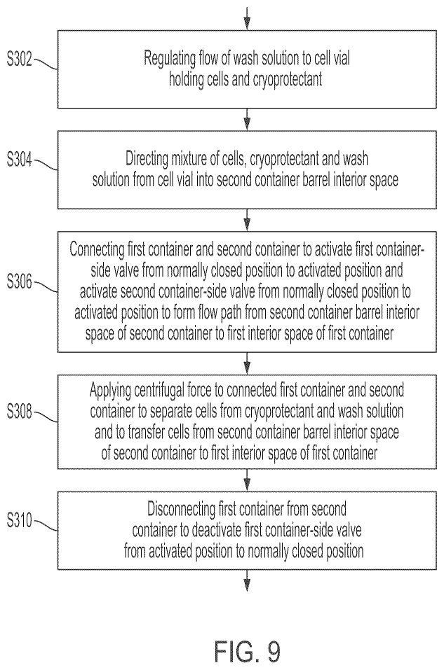

A process for preparing a cell solution for delivery such as a cell-based cell solution for delivery will be described with reference to FIG. 9.

The process for preparing the cell solution for delivery may include a step S302 of regulating, by the flow regulator 302, a flow of wash solution from the source of wash solution to the cell vial 308 (e.g., see FIG. 8) holding a mixture of cells and cryoprotectant. The wash solution may serve to wash the cells that were previously cryopreserved in the cryoprotectant. Regulation of the wash solution may serve to prevent osmotic shock to the cells contained in the cell vial 308.

The process for preparing the cell solution for delivery may include a step S304 of fluidly directing a mixture of the cells, the cryoprotectant and the wash solution from the cell vial 308 through the second flow path control module 232 into the second container barrel interior space 212 of the third section 208C of the second container outer body 208.

The process for preparing the cell solution for delivery may include a step S306 of connecting the first container 100 and the second container 200. Connecting the first container 100 and the second container 200 may include aligning the first tube body axis A and the second tube body axis C, and engaging the male thread of the first container outer body 108 with the female thread of the first section 208A of the second container outer body 208 through rotation of the first container 100 relative to the second container 200 to advance the first container-side valve 126 towards the second container-side valve 220. As the first container-side valve 126 continues to advance towards the second container-side valve 220, the tapered portion of the valve stem 224 closest to the first seal 132 advances past the first valve casing 128 to contact and activate the first seal 132 from the normally closed position to the activated position.

As the male thread of the first container outer body 108 continues to engage the female thread of the first section 208A to advance the first container-side valve 126 towards the second container-side valve 220, the first valve casing 128 of the first container-side valve 126 may be brought into contact with the valve cap 222. For example, the first valve casing 128 may be brought into contact with the attachment surface 222B of the valve cap 222. A force in a direction of the first container 100 towards the second container 200 resulting from the advancement of the first container-side valve 126 towards the second container-side valve 220 may be transferred from by the first valve casing 128 to the valve cap 222 to move the valve cap 222 having the valve stem 224 attached thereto along the predetermined range of the second tube body axis C in the direction of the second end 204B of the second tube body 204 towards the second seal 226. As the valve cap 222 is moved towards the second seal 226, the tapered portion of the first portion 224A of the valve stem 224 may contact and activate the second seal 226 from the normally closed position to the activated position. In a state where the valve cap 222 is moved to an end of the predetermined range closest to the second end 204B of the second tube body 204, the tapered portions of the valve stem 224 contact the first seal 132 and the second seal 226, respectively, to activate the first container-side valve 126 and the second container-side valve 220 to form a pathway for flow from the second interior space 206 of the second tube body 204 to the first interior space 106 of the first tube body 104.

It is noted that prior to connecting the first container 100 and the second container 200, the first interior space 106 of the first container 100 is pre-loaded with a predetermined volume of cell delivery solution according to step S110, as described above.

The predetermined volume of the cell delivery solution to be loaded into the first interior space 106 may be selected based on target dose volume to be prepared. For example, the predetermined volume of the cell delivery solution may be selected to be the same as the volume of the first interior space 106. By selecting the predetermined volume of the cell delivery solution to be the same as the volume of the first interior space 106, any additional loading of cells from the second container 200 into the first interior space 106 may displace the same or substantially the same volume of the cell delivery solution such that the first interior space 106 having the cells and the cell delivery solution (together to be considered a prepared dose) will occupy the entirety or substantially the entirety of the first interior space 106. In another example, the predetermined volume of the cell delivery solution may be selected such that the predetermined volume of the cell delivery solution with a predetermined volume of the cells loaded from the second container 200 into the first interior space 106 will occupy the entirety or substantially the entirety of the first interior space 106.

The process for preparing the cell solution for delivery may include a step S308 of applying a centrifugal force to the connected first container 100 and second container 200. The step S308 may include loading the connected first container 100 and second container 200 into a centrifuge in an orientation such that during the spinning operation of the centrifuge along a spin axis, at least a portion of the cells in the second container 200 moves outward in the radial direction of the spin axis from the second container 200 to the first container 100. Since the first container-side valve 126 and the second container-side valve 220 are each in the activated position after the step S306, the at least a portion of the cells in the second container 200 is moved from the second container barrel interior space 212 through the second seal 226 that is in the activated position, through the valve stem 224, through the first seal 132 that is in the activated position and into the first interior space 106 defined by the first container barrel 110 to form a dose of a cell solution for delivery.

In the step S308, the centrifuge can be controlled to operate for at least a predetermined length of time, to operate at a predetermined speed, or both to ensure that a sufficient number of cells are separated from the cryoprotectant and wash solution in the first container 100 and moved to the first interior space 106 of the first container barrel to produce a dose having a cell concentration that meets a predetermined target concentration and volume.

The process for preparing the cell solution for delivery described above may be modified to include other steps. For example, after step S304 directing the mixture of cells, cryoprotectant and wash solution from cell vial 308 into the second container barrel interior space 212 and before performing step S306 of connecting the first container 100, the process for preparing the cell solution for delivery may include a step of applying centrifugal force to the mixture of the cells, cryoprotectant and wash solution contained in the second container 200 to separate the cells from the cryoprotectant and wash solution. The cells, separated from the cryoprotectant and wash solution, may be formed as a pellet primarily in a portion of the second container barrel interior space 212 in the third part 212E of the third section 208C of the second container outer body 208. After forming the pellet of cells in the third part 212E of the third section 208C of the second container outer body 208, the step S306 is performed to apply centrifugal force to transfer the cells from the second container 200 to the first container 100 via the first flow path control module 124 having the first container-side valve 126 in the activated position and the second container-side valve 220 in the activated position. Performing the step of applying centrifugal force to the mixture of the cells, cryoprotectant and wash solution contained in the second container 200 before perform the step S308 may minimize the amount of cryoprotectant and wash solution transferred to the first container 100 in the step S308 and may increase the number of cells transferred to the first container 100 in the step S308 to thereby increase the likelihood that a cell concentration of the dose prepared in the first container 100 meets a predetermined target concentration.

The process for preparing the cell solution for delivery may include a step S310 of disconnecting the first container 100 from the second container after the step S308 to deactivate the first container-side valve 126 and the second container-side valve 220. The first container 100 and the second container 200 may be disconnected by disengaging the male thread of the first container outer body 108 from the female thread of the first section 208A of the second container outer body 208 through counter-rotation of the first container 100 relative to the second container 200 to retract the first container-side valve 126 away from the second container-side valve 220. As the first container-side valve 126 continues to retract from the second container-side valve 220, the tapered portion of the second portion 224B of the valve stem 224 that activated the first container-side valve 126 is retracted away from the first seal 132 to deactivate the first seal 132 from the activated position to the normally closed position to thereby reduce or completely eliminate flow through the first seal 132 of the first container-side valve 126.

Upon completion of the step S310, a dose of cells including the mixture of the cells and cell delivery solution are enclosed in the first interior space 106 of the first container barrel 110 by the first container-side valve 126 in the normally-closed position.

According to some embodiments of the process for preparing the cell solution for delivery, the cells originally cryopreserved in the cryoprotectant (i.e., a formulated cell drug product) and held by the cell vial 308 are washed and transferred from the cell vial 308 to the second container 200 and further to the first container 100 in a flow path that minimizes or eliminates a risk of contamination of the cells through contact with an external environment. Therefore, the cell solution for delivery can be considered to be aseptic or near aseptic.

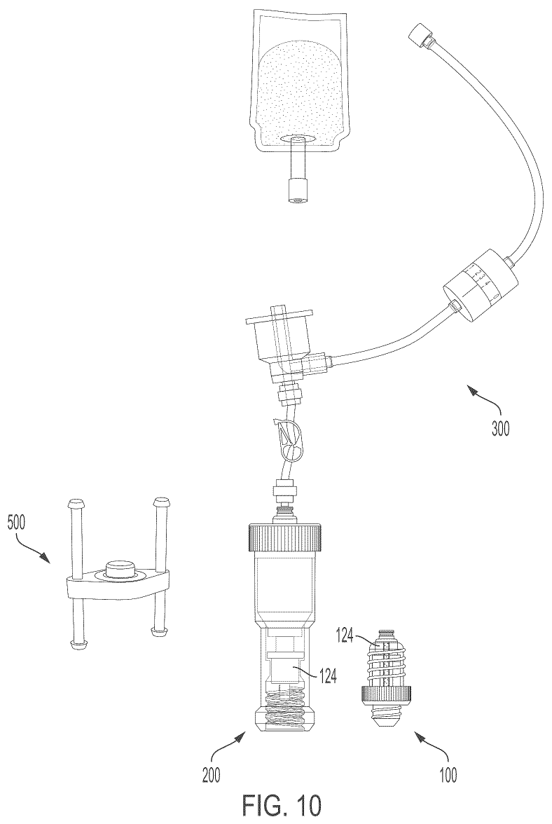

FIG. 10 illustrates an example of a medical device system 10 for implementing dose preparation according to one embodiment. The medical device system 10 may include a first container 100 (also referred to as a dose preparation tube), a second container 200 (also referred to as a wash tube) a second container loading module 300, a movable body 400 and a first container mixing module 500.

The medical device system 10 illustrated in FIG. 10 is similar to the medical device system 10 illustrated in FIG. 1, but further includes the movable body 400 and the first container mixing module 500. The features of first container 100, second container 200 and second container loading module 300 shown in FIG. 10 are similar to the features of first container 100, second container 200 and second container loading module 300 described above with reference to FIGS. 1-9.

### Movable Body

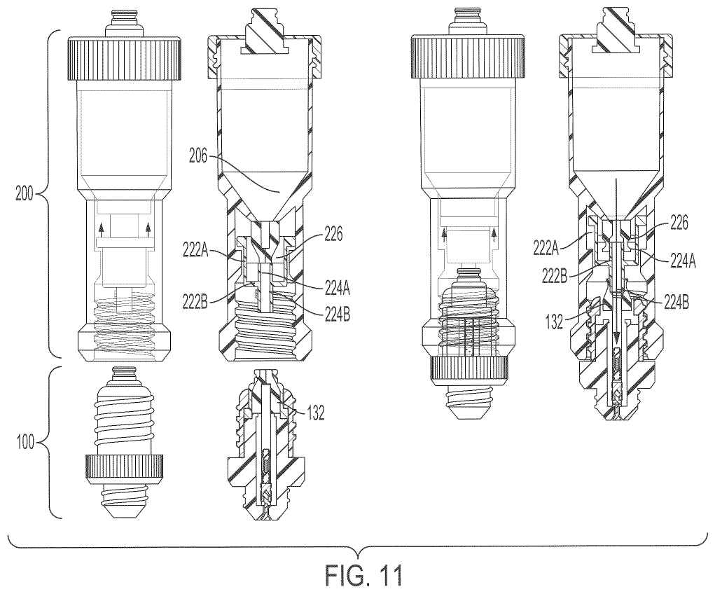

FIGS. 11, 12 and 13A-D illustrate a movable body 400 arranged in first interior space 106 of first container 100.

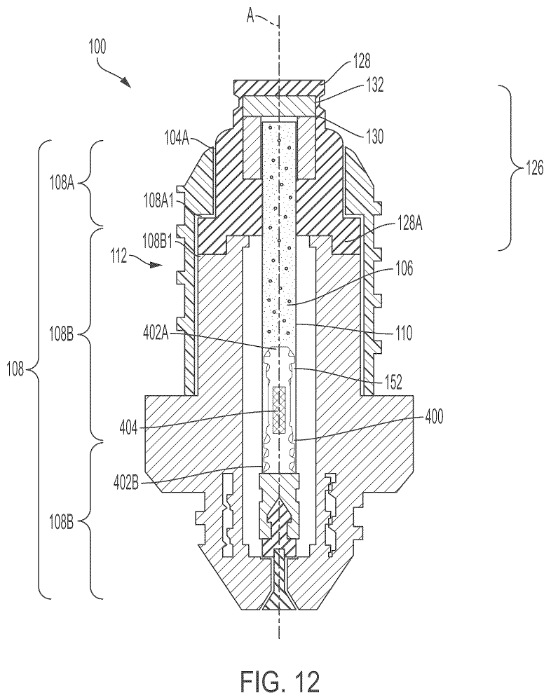

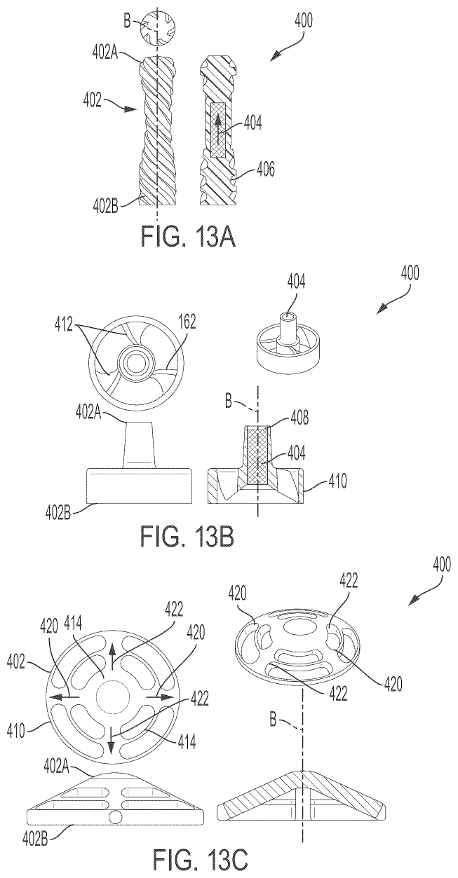

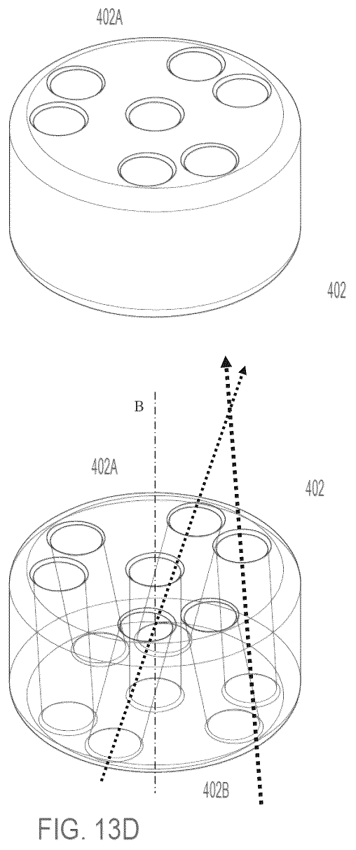

Referring to FIGS. 12 and 13A-D, a movable body 400 that may be used with the first container 100 will be described. For ease of explanation, a description of a single movable body 400 is provided. However, a plurality of movable bodies 400 is also contemplated.

The movable body 400 may have a geometry or structure to allow the movable body 400 be moved within the first interior space 106 of the first container barrel 110 by an external force. Movement of the movable body 400 is not particularly limited. In one example, the movable body 400 may have a geometry or structure to allow the movable body 400 to move in parallel with or on the first tube body axis A within the first container barrel 110. A range of movement of the movable body 400 in parallel with or on the first tube body axis A can include movement between the two ends of the first container barrel 110 or a shorter range. In another example, the movable body 400 may have a geometry or structure to allow the movable body 400 to rotate on the first tube body axis A within the first container barrel 110. In another example, the movable body 400 may have a geometry or structure to allow the movable body 400 to both move in parallel with or on the first tube body axis A and to rotate on the first tube body axis A within the first container barrel 110.

The movable body 400 may be configured to be moved in the range of movement by an external force. The external force is not particularly limited. In an example, the movable body 400 may be configured to be moved by manually or mechanically moving the first container 100 relative to the movable body 400. In another example, the movable body 400 may be configured to be moved by a magnetic force.

The movable body 400 may include a movable body casing 402 and one or more magnets 404 (or magnetic material) arranged to the movable body casing 402. As described herein, magnetic material may be a suitable alternative to magnets. For example, if a movable body as disclosed herein is a magnetic material, a magnet external to the apparatus disclosed herein may be used to achieve movement of the magnetic material.

A first example of the movable body 400 will be described with reference to FIG. 13A.

In the first example, the movable body casing 402 may extend along a length axis B between a first end 402A of the movable body casing 402 and a second end 402B of the movable body casing 402. A maximum diameter of the movable body casing 402 along the length axis B may be selected to be less than a minimum diameter of the first interior space 106 along the first tube body axis A.

An outer diameter of a first portion of the movable body casing 402 provided between the first end 402A of the movable body casing 402 and the second end 402B of the movable body casing 402 may be less than an outer diameter of a second portion of the movable body casing 402 between the first end 402A of the movable body casing 402 and the first portion of the movable body casing 402. The outer diameter of the first portion of the movable body casing 402 may be less than an outer diameter of a third portion of the movable body casing 402 between the second end 402B of the movable body casing 402 and the first portion of the movable body casing 402.

At least a portion of the external surface of the movable body casing 402 may be formed as a cylindrical shaft with helical flutes 406.

Further, the movable body casing 402 may encase the magnet 404. The magnet 404 may be encased within the movable body casing 402 to orient a magnet polarity of the magnet 404 to be aligned with or parallel with the length axis B of the movable body casing 402 to allow a magnetic force applied to the magnet 404 to be transmitted to the movable body casing 402 to move the movable body 400 within the first container barrel 110 in parallel with or along the first tube body axis A.

A second example of the movable body 400 will be described with reference to FIG. 13B.

In the second example, the movable body casing 402 may extend along the length axis B between a first end 402A of the movable body casing 402 and a second end 402B of the movable body casing 402. A maximum diameter of the movable body casing 402 along the length axis B may be selected to be less than a minimum diameter of the first interior space 106 along the first tube body axis A.

The movable body casing 402 may include an inner cylinder 408 extending along the length axis B and an outer cylinder 410 extending along the length axis B. The inner cylinder 408 and the outer cylinder 410 may be coaxial along the length axis B.

The movable body casing 402 may further include a plurality of blades 412 connecting the inner cylinder 408 and the outer cylinder 410. The plurality of blades 412 may be arranged to be spaced apart from each other circumferentially around the length axis B. The plurality of blades 412 may be arranged to be spaced apart from each other in an equidistant manner circumferentially around the length axis B. The plurality of blades 412 may be arranged to have rotational symmetry relative to the length axis B.

The magnet 404 may be encased within the inner cylinder 408 of the movable body casing 402 to orient a magnet polarity of the magnet 404 to be parallel with or substantially parallel with the length axis B of the movable body casing 402 to allow a magnetic force applied to the magnet 404 to be transmitted to the movable body casing 402 to move the movable body 400 within the first container barrel 110 along the first tube body axis A. Depending on the magnetic force applied to the magnet 404, the movable body 400 may be moved in parallel with or along the first tube body axis A, moved to rotate about the first tube body axis A or both.

The movable body 400 may be arranged within the first container barrel 110 such that a portion of the inner cylinder 408 protrudes past the outer cylinder 410 along the length axis B towards the first opening 104A of the first tube body 104 to be closer to the first opening 104A of the first tube body 104 than the outer cylinder 410.

In such an arrangement, a side of the movable body 400 closer to the second opening 104B of the first tube body 104 may have a concave geometry. Further a side of the movable body 400 closer to the first opening 104A of the first tube body 104 may have a convex geometry, and a side of the first container-side valve 126 facing the side of the movable body 400 having the convex geometry may have a concave geometry fitted to the convex geometry of the side of the movable body 400. The fitted geometries of the first container-side valve 126 and the movable body 400 may increase a range of movement of the movable body 400 in the first interior space 106 of the first container barrel 110 along the first tube body axis A.

A third example of the movable body 400 will be described with reference to FIG. 13C.

In the third example, the movable body casing 402 may extend along a length axis B between a first end 402A of the movable body casing 402 and a second end 402B of the movable body casing 402. A maximum diameter of the movable body casing 402 along the length axis B may be selected to be less than a minimum diameter of the first interior space 106 along the first tube body axis A.

The movable body casing 402 may include an inner disc 414, a plurality of outer rings and a plurality of connectors connecting the plurality of outer rings to each other and the inner disc 414.

The inner disc 414 may be arranged such that the length axis B passes through a center of the inner disc 414. The plurality of outer rings may include an outer ring 416 and an inner ring 418. A diameter of the outer ring 416 may be greater than a diameter of the inner disc 414. A diameter of the inner ring 418 may be greater than the diameter of the outer ring 416. The inner disc 414, the outer ring 416 and the inner ring 418 may be arranged coaxially along the length axis B. The inner disc 414, the outer ring 416 and the inner ring 418 may be arranged in a tiered manner. The inner disc 414 may be arranged on a first plane along the length axis B, the inner ring 418 may be arranged on a second plane along the length axis B and the outer ring 416 may be arranged on a third plane along the length axis B, where the first, second and third planes are perpendicular to the length axis B, and where the second plane is provided between the first plane and the third plane.

The plurality of connectors may include a first pair of connectors 420 and a second pair of connectors 422. The first pair of connectors 420 may be provided on a fourth plane on which the length axis B is provided. The second pair of connectors 422 may be provided on a fifth plane on which the length axis B is provided, where the fifth plane is perpendicular to the fourth plane.

One connector of the first pair of connectors 420 may be provided to connect the inner disc 414, the outer ring 416 and the inner ring 418 in one direction provided on the fourth plane, and another connector of the first pair of connectors 420 may be provided to connect the inner disc 414, the outer ring 416 and the inner ring 418 in another direction provided on the fourth plane, where the two directions on the fourth plane have reflectional symmetry with respect to the fifth plane.

One connector of the second pair of connectors 422 may be provided to connect the inner disc 414, the outer ring 416 and the inner ring 418 in one direction provided on the fifth plane, and another connector of the second pair of connectors 422 may be provided to connect the inner disc 414, the outer ring 416 and the inner ring 418 in another direction provided on the fifth plane, where the two directions on the fifth plane have reflectional symmetry with respect to the fourth plane.

When the movable body 400 is viewed along the length axis B, a plurality of gaps are defined between the outer ring 416 and inner ring 418 by portions of the outer ring 416 and the inner ring 418 not connected by the first pair of connectors 420 and the second pair of connectors 422, and a plurality of gaps are defined between the inner ring 418 and the inner disc 414 by portions of the inner ring 418 and the inner disc 414 not connected by the first pair of connectors 420 and the second pair of connectors.

A plurality of magnets 404 may be encased in the first pair of connectors 420 and the second pair of connectors 422. A first magnet of the plurality of magnets 404 may be encased within the one connector of the first pair of connectors 420 to orient a magnet polarity of the first magnet to be aligned with or parallel with the one direction provided on the fourth plane. A second magnet of the plurality of magnets 404 may be encased within the another connector of the first pair of connectors 420 to orient a magnet polarity of the second magnet to be aligned with or parallel with the another direction provided on the fourth plane. A third magnet of the plurality of magnets 404 may be encased within the one connector of the second pair of connectors 422 to orient a magnet polarity of the third magnet to be aligned with or parallel with the one direction provided on the fifth plane. A fourth magnet of the plurality of magnets 404 may be encased within the another connector of the second pair of connectors 422 to orient a magnet polarity of the fourth magnet to be aligned with or parallel with the another direction provided on the fifth plane. The magnet polarity of each of the first to fourth magnets 404 allow a magnetic force applied to the magnets 174 to be transmitted to the movable body casing 402 to move the movable body 400 within the first container barrel 110 along the first tube body axis A. Depending on the magnetic force applied to the magnets 404, the movable body 400 may be moved in parallel with or along the first tube body axis A, moved to rotate about the first tube body axis A or both.

The movable body 400 may be arranged within the first container barrel 110 such that the inner disc 414 is positioned to be closer to the first opening 104A of the first tube body 104 than the inner ring 418, and the outer ring 416 is positioned to be closer to the second opening 104B of the first tube body 104 than the inner ring 418.

In such an arrangement, a side of the movable body 400 closer to the first opening 104A of the first tube body 104 may have a convex geometry, and a side of the first container-side valve 126 facing the side of the movable body 400 having the convex geometry may have a concave geometry fitted to the convex geometry of the side of the movable body 400. The fitted geometries of the first container-side valve 126 and the movable body 400 may increase a range of movement of the movable body 400 in the first interior space 106 of the first tube body 104 along the first tube body axis A.

A fourth example of the movable body 400 will be described with reference to FIG. 13D.

In the fourth example, the movable body casing 402 may extend along a length axis B between a first end 402A of the movable body casing 402 and a second end 402B of the movable body casing 402. A maximum diameter of the movable body casing 402 along the length axis B may be selected to be less than a minimum diameter of the first interior space 106 along the first tube body axis A.

The movable body casing 402 defines two or more through-holes or passages extending between the first end 402A and the second end 402B. As shown in FIG. 13D, each of the two or more through-holes extends between the first end 402A and the second end 402B along an axis that is parallel or oblique to length axis B. Further, the axis of each of the two or more through holes can intersect such that flows of fluid passing through the two or more through holes between the first end 402A and the second end 402B are directed to each other and is encouraged to mix as the movable body 400 moves along the first tube body axis A.

The movable body 400 may be formed as a solid body by a material such as stainless steel having the two or more through-holes defined therein. The movable body 400 can be made to move within the first container barrel 110 by, for example, changing the orientation of the first container barrel 110 and allowing the weight of the movable body 400 to be moved by gravity along the first tube body axis A. Other material that is sufficiently dense and heavy to allow for gravitational mixing is also contemplated.

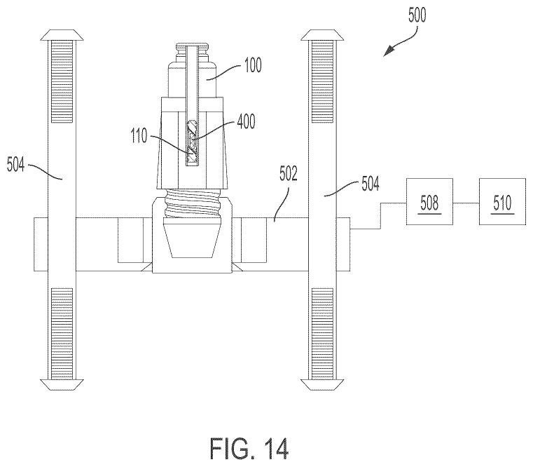

Referring to FIG. 14, the first container mixing module 500 may include one or more external magnets 502 and one or more movement guides 504.

The movement guide 504 may be connected to one or both of the first container 100 and the one or more external magnets 502. Further, the movement guide 504 may move the first container 100 relative to the one or more external magnets 502 to direct the magnet polarity of the one or more external magnets 502 to act on the one or more magnets 404 of the movable body 400 to move the movable body 400 within the first interior space 106. As described above, movement of the movable body 400 within the first interior space 106 may include movement of the movable body 400 in parallel with or on the first tube body axis A and rotation of the movable body 400 on the first tube body axis A within the first container barrel 110. Movement of the movable body 400 within the first interior space 106 mixes the cells in the pellet with the cell delivery matrix to prepare a dose.

In an example, the first container mixing module 500 can include a stand 506 on which the first container barrel 110 is removably placed and/or attached. Further, the movement guide or guides 504 are arranged relative to the stand 506, on which the first container barrel 110 is removably placed and/or attached, to guide the external magnet or magnets 502 relative to the first container barrel 110 to direct the magnet polarity of the external magnet or magnets 502 to act on the one or more magnets 404 of the movable body 400 to move the movable body 400 within the first interior space 106.

The first container mixing module 500 may include an actuator 508 and a controller 510. The controller 510 may control the actuator 508 to move the external magnet or magnets 502 guided by the movement guide or guides 504 to direct the magnet polarity of the external magnet or magnets 502 to act on the one or more magnets 404 of the movable body 400 to move the movable body 400 within the first interior space 106.

Referring back to FIG. 9, the process for preparing a cell solution for delivery may include placing and/or attaching the first container 100 to which cells have been transferred (in step S308) to the stand 506. Further, the process may include moving the external magnet or magnets 502 manually or by controller 510 and actuator 508 to act on the one or more magnets 404 of the movable body 400 to move the movable body 400 within the first interior space 106.

The movement of the one or more magnets 404 may reduce the level of settlement of the cells within the first interior space 106 and promote suspension of the cells in the cell delivery solution as a heterogeneous mixture.

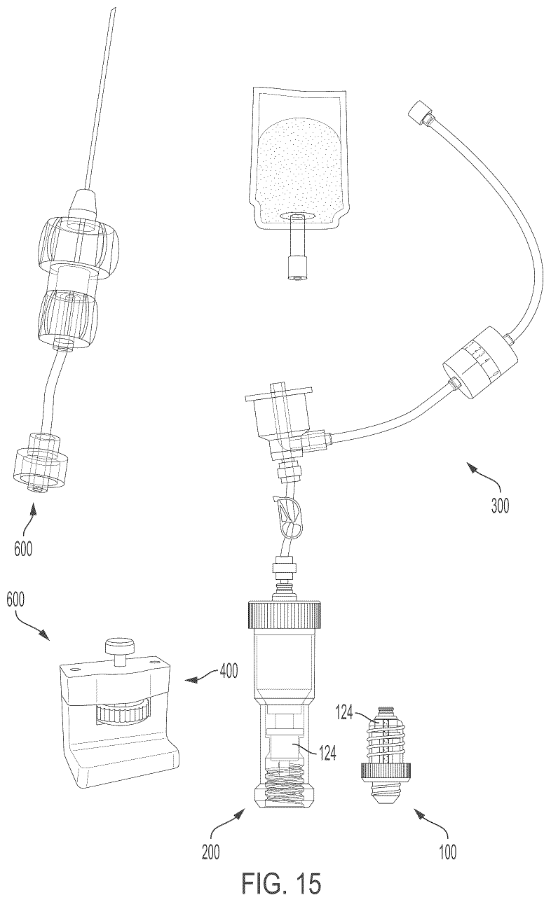

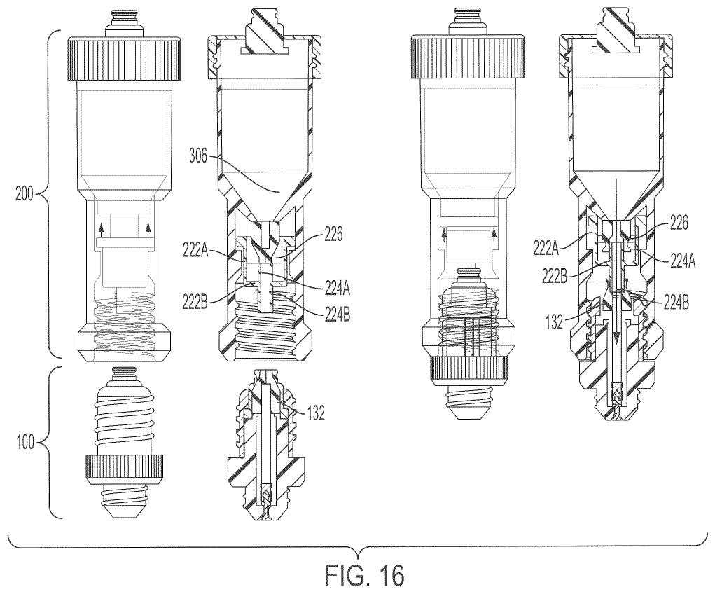

FIG. 15 illustrates an example of a medical device system 10 for implementing dose preparation according to one embodiment. The medical device system 10 may include a first container 100 (also referred to as a dose preparation tube), a second container 200 (also referred to as a wash tube) and a second container loading module 300 and a dose loading module 600.

The medical device system 10 illustrated in FIG. 15 is similar to the medical device system 10 illustrated in FIG. 1, but further includes the dose loading module 600. The features of first container 100, second container 200 and second container loading module 300 shown in FIG. 10 are similar to the features of first container 100, second container 200 and second container loading module 300 described above with reference to FIGS. 1-9.

### Dose Loading System

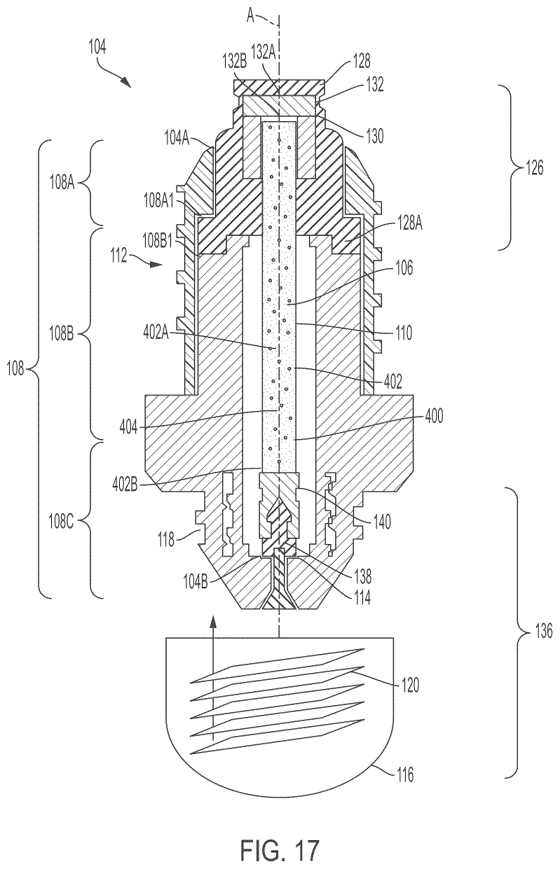

Referring to FIGS. 17-19, the dose loading module 600 for loading a dose prepared by mixture of cells and the cell delivery matrix in the first container 100 into a cell delivery device will be described.

The first container outer body 108 may have a geometry or structure that allows the first container 100 to receive a plunger of a plunger system (described in more detail below), whereby the plunger moves relative to the first container 100 to reduce the volume of the first interior space 106 between the plunger seal 140 and the first opening 104A of the first tube body 104. As an example, the first container outer body 108 may define a through hole 114 for receiving the plunger. The through hole 114 may be provided at the second end of the first tube body 104.

The first container outer body 108 may have a geometry or structure for directly or indirectly connecting the first container 100 to a cover 116. The cover 116 may be removably connected to the first container outer body 108 to cover the through hole 114. For example, the cover 116 may be connected to the first container 100, which in turn can be connected to the second container 200. The first container 100 and the cover 116, when connected together, may have a geometry or structure that allows the first container 100 and the cover 116 to be fitted within the compartment of the centrifuge. The first container outer body 108 may include a first part 118 of a connection or coupling mechanism to directly or indirectly connect the first container 100 to the cover 116. As an example, the first container outer body 108 may include one part of a threaded connection. The cover 116 may include a second part 120 of the connection or coupling mechanism to directly or indirectly connect the cover 116 to the first container 100. As an example, the cover 116 may include another part of the threaded connection that detachably connects the cover 116 to the first container 100. The one part of the threaded connection provided to the first container outer body 108 may be one of a male thread (as shown in the drawings) and a female thread, while the another part of the thread connection provided to the cover 116 may be the other of the male thread and the female thread (as shown in the drawings).

The first container barrel 110 may be arranged within the first container outer body 108 along the first tube body axis A. The first container barrel 110 may define the first interior space 106. The first interior space 106, defined by the first container barrel 110, may be connected to the first opening 104A at the first end of the first tube body 104. Further, the first interior space 106, defined by the first container barrel 110, may be connected to a second opening 104B at the second end of the first tube body 104. For example, the first interior space 106 may be connected to the second opening 104B at the second end of the first tube body 104 to be in communication with the through hole 114 for receiving the plunger. For example, the communication between the first interior space 106 and the through hole 114 may be configured to allow a distal end of the plunger to be moved past the through hole 114 and into the first interior space 106, as described in more detail below.

The dimensions of the first container barrel 110 is not particularly limited. For example, a length of the first container barrel 110 along the first tube body axis A and an inner diameter of the first container barrel 110 in a direction perpendicular to the first tube body axis A may be selected to allow a portion of the plunger system to be moved within the first container barrel 110 to change a volume of the first interior space 106.

The dose loading module 600 may include a plunger system 136. The plunger system 136 may be configured to form a seal with the interior surface of the first container barrel 110 to reduce or prevent flow past the second opening 104B of the first tube body 104 and the through hole 114. The geometry and structure of the plunger system 136 is not particularly limited.

As an example, the plunger system 136 may include a plunger head 138 and a plunger seal 140. The plunger head 138 and the plunger seal 140 may be provided as a unitary structure or separate structures.

The plunger head 138 may be arranged within the first container barrel 110. Further the plunger seal 140 may be attached to the plunger head 138 to form a seal with the interior surface of the first container barrel 110 to reduce or prevent flow past the second opening 104B of the first tube body 104 and the through hole 114. Still further, the plunger head 138 and the plunger seal 140 attached to the plunger head 138 may be configured to advance within the first interior space 106 of the first container barrel 110 toward the first opening 104A of the first tube body 104 and to be retracted within the interior space 106 of the first container barrel 110 toward the second opening 104B of the first tube body 104 by forces applied thereto while maintaining the seal with the interior surface of the first container barrel 110. Advancement of the plunger head 138 and the plunger seal 140 reduces the volume of the first interior space 106 between the plunger seal 140 and the first opening 104A while retraction of the plunger head 138 and the plunger seal 140 increases the volume of the first interior space 106 between the plunger seal 140 and the first opening 104A.

As an example, the plunger system 136 may include a plunger 142. The plunger 142 may be in the form of a wire that is configured to be inserted through the through hole 114 of the first tube body 104. The plunger head 138, the plunger seal 140 and the plunger 142 may be provided as a unitary structure or separate structures. A distal end of the plunger 142 may be inserted through the through hole 114 of the first tube body 104 to directly or indirectly transfer a force applied to the plunger 142 to the plunger head 138 and plunger seal 140 to advance the plunger head 138 and the plunger seal 140 within the first interior space 106 within the first container barrel 110 toward the first opening 104A of the first tube body 104 and to retract the plunger head 138 and the plunger seal 140 within the first interior space 106 defined by the first container barrel 110 toward the second opening 104B of the first tube body 104

In an embodiment shown in S, in which the first container 100 is provided with the plunger head 138 and the plunger seal 140 arranged in the first interior space 106, a third outer body part 108C of the first container outer body 108 can define the through hole 114 that allows for the plunger 142 to be passed therethrough to transfer a force to the plunger head 138 and the plunger seal 149 to move the plunger head 138 and the plunger seal 140.

In an embodiment shown in FIGS. 2 and 3 in which the first container 100 is not provided with the plunger head 138 and the plunger seal 140 arranged in the first interior space 106, the first container outer body 108 does not define the through hole 114 and may include a surface to seal an end of the first interior space 106 opposite to the end of the first interior space 106 to which the first container-side valve 126 is provided.

Referring to FIG. 4, the process for assembling the first container 100 may include a step of arranging the plunger head 138 having the plunger seal 140 attached thereto in the first container barrel 110 to form a seal with the interior surface of the first container barrel 110.

The process for assembling the first container 100 may include, after the step of arranging the plunger head 138 and the plunger seal 140 in the first container barrel 110, a step of connecting the third outer body part 108C to the second outer body part 108B by, for example, press fitting, adhesive and/or a threaded connection to provide access to the plunger head 138 through the through hole 114 defined by the third outer body part 108C.

The dose loading module 600 may further include a third container 602. The third container 602 may include a male luer 604 and a Tuohy-Borst adapter 606 fluidly connected to the male luer 604.

The male luer 604 may include another part of the threaded connection that detachably connects the male luer 604 to the one part of the threaded connection on the outer surface of the first valve casing 128 to thereby provide a pathway for flow from the first interior space 106 to the Tuohy-Borst adapter 606. The Tuohy-Borst adapter 606 is configured to receive a distal end of a needle 608 of a dose delivery device.

The stand 506 may hold the plunger 142 of the plunger system 136 such that the distal end of the plunger 142 is guided into the through hole 114 of the first tube body 104 placed on the stand 506 to contact the plunger head 138 of the plunger system 136.

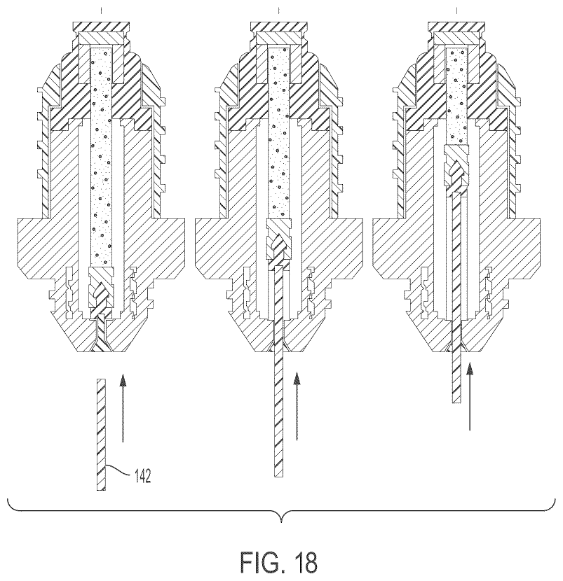

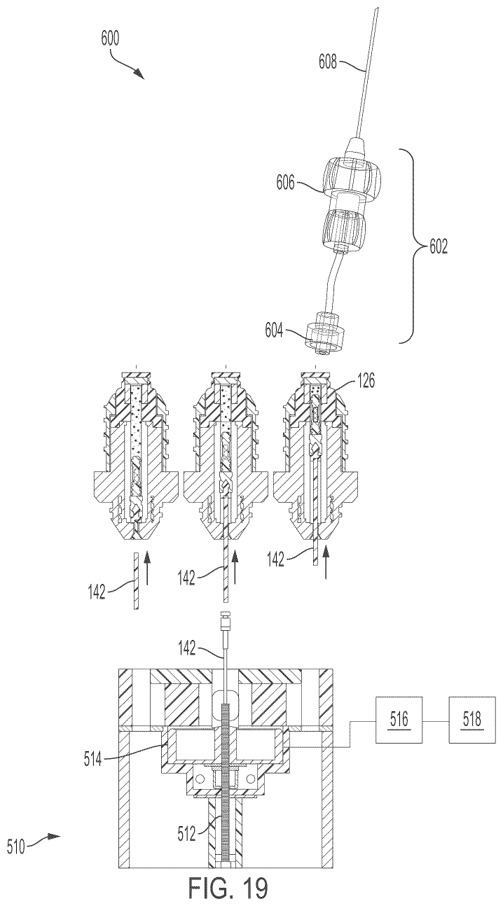

The dose loading module 600 may further include a dispensing device 610. The dispensing device 610 may be arranged to the stand 506 of the first container mixing module 500. As shown in FIGS. 18 and 19, the dispensing device 610 may apply a force to the plunger 142 of the plunger system 136 to advance the plunger head 138 and the plunger seal 140 towards the first container-side valve 126 to reduce the volume of the first interior space 106. The reduction in the volume of the first interior space 106 causes a flow of the dose in the first interior space 106 through the pathway formed by the connection of the first container-side valve 126 and the male luer 604 to load the dose or a portion of the dose into the dose delivery device through the distal end of the needle 608 arranged within the Tuohy-Borst adapter 606.

The dispensing device 610 may include a lead screw 612 and a dispensing wheel 614. The dispensing wheel 614 may be connected to the plunger 142. Further, the dispensing wheel 614 may be movably connected to a thread of the lead screw 612. Rotation of the dispensing wheel 614 causes the dispensing wheel 614 to move along a longitudinal axis of the lead screw 612 to move the plunger 142 to advance the plunger head 138 and the plunger seal 140 towards the first container-side valve 126 to reduce the volume of the first interior space 106. As described above, the reduction in the volume of the first interior space 106 causes a flow of the dose in the first interior space 106 through the pathway formed by the connection of the first container-side valve 126 and the male luer 604 to load the dose or a portion of the dose into the dose delivery device through the distal end of the needle 608 arranged within the Tuohy-Borst adapter 606.

The dispensing device 610 may further include an actuator 616 and a controller 618. The controller 618 may control the actuator 616 to rotate the dispensing wheel 614 to cause the dispensing wheel 614 to move along longitudinal axis of the lead screw 612 to move the plunger 142 to advance the plunger head 138 and the plunger seal 140 towards the first container-side valve 126 to reduce the volume of the first interior space 106. As described above, the reduction in the volume of the first interior space 106 causes a flow of the dose in the first interior space 106 through the pathway formed by the connection of the first container-side valve 126 and the male luer 604 to load the dose or a portion of the dose into the dose delivery device through the distal end of the needle 508 arranged within the Tuohy-Borst adapter 606.

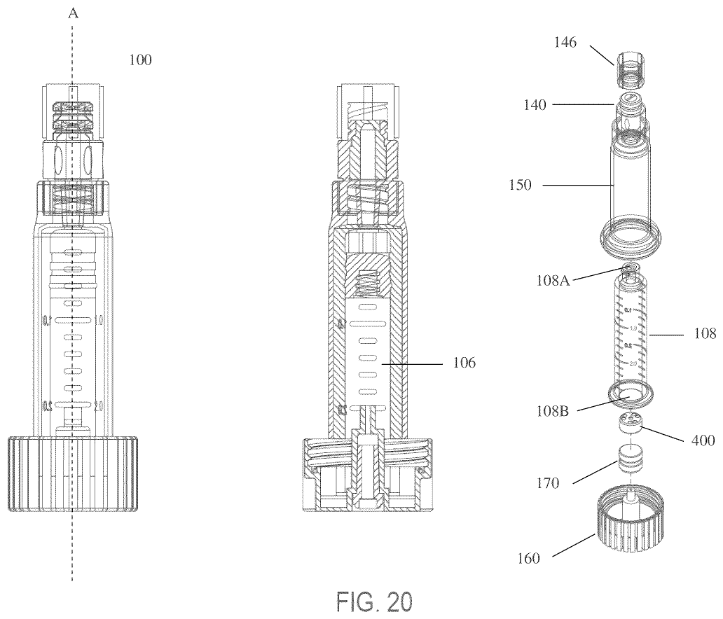

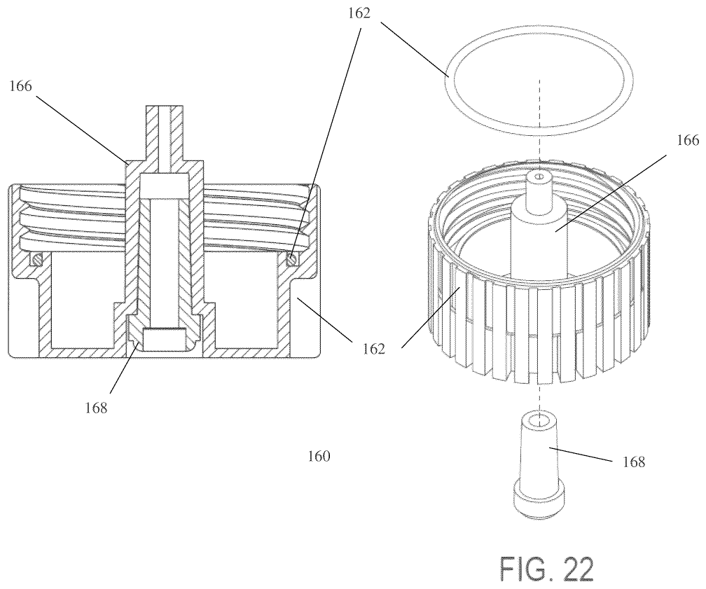

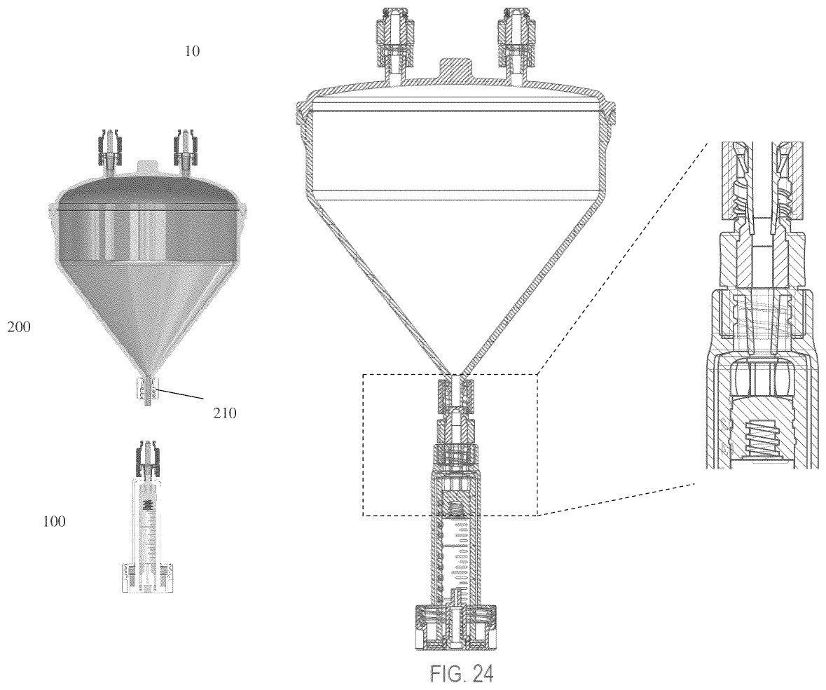

FIGS. 20-25 illustrate an example of a medical device system 10 (see FIG. 24) for implementing dose preparation according to one embodiment. The medical device system 10 may include a first container 100 (also referred to as a dose preparation tube) and a second container 200 (also referred to as a wash tube) that is detachably connected to the first container 100.

### First Container (Dose Preparation Tube)

The first container 100, that is an apparatus, will now be described with reference to FIG. 20. The first container 100 may include a first container outer body 108. The first container outer body 108 may extend along a first tube body axis A between a first end of the first tube outer body 108 and a second end of the first container outer body 108.

The first container outer body 108 may define a first interior space 106. The first interior space 106 may be connected to a first opening 108A at the first end of the first container outer body 108 and a second opening 108B at the second end of the first container outer body 108.

The volume of the first container outer body 108 is not particularly limited. For example, the volume of the first interior space 106 may be selected based on a target dose volume of a dose to be prepared. For example, the volume of the first interior space 106 may be about 1.8 mL or 2.0 mL.

A material or materials for forming the first container outer body 108 is not particularly limited. Examples of the material or materials for forming the first container outer body 108 may include low leachable and/or extractable materials such as cyclic olefin polymer, cyclic olefin copolymer, perfluoroalkoxy alkane, ethylene tetra fluoroethylene, cyclic block copolymers, polyethylene, polypropylene and polycarbonates. The material or materials for forming the first container may also be selected based on contemplated methods of manufacture, such as injection molding.

The first container 100 may further include a first container-side valve 140 and a sheath 150.

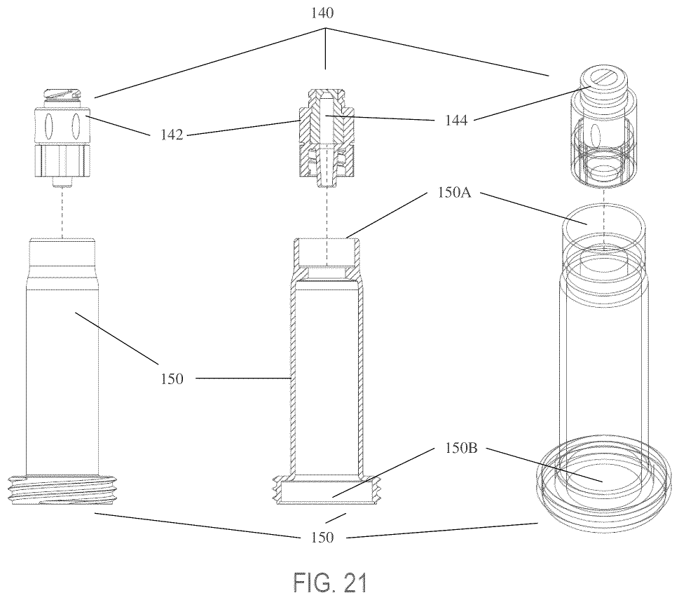

Referring to FIGS. 20 and 21, the sheath 150 may define a sheath interior space 152 in which at least a portion of the first container outer body 108 can be removably inserted. The sheath interior space 152 may be connected to a first opening 150A at a first end of the sheath 150 and a second opening 108B at a second end of the sheath 150. In some embodiments, a sheaths provides or maximizes aseptic condition or means to protect against contamination of the contents of the first and/or second container.

In some embodiments, the sheath 150 comprises a material that can be sterilized. Non-limiting examples of materials that can be sterilized include polycarbonate, polystyrene, nylon, polyethylene, cyclic olefin copolymer, CoPC, and acrylic

The first container-side valve 140 may be connected to the sheath 150 at the first opening 150A of the sheath 150. In a configuration in which the first container outer body 108 is inserted within the sheath interior space 152, the first opening 108A of the first container outer body 108 is communicated with the first container-side valve 140.

The first container-side valve 140 may include a valve casing 142 connected to the sheath 150 at the first opening 150A of the sheath 150. The first container-side valve 140 may further include a seal 144 arranged within the valve casing 142. The seal 144 is configured to provide a normally closed position and an activated position (see FIG. 24) of the first container-side valve 140. The structure of the seal 144 is not particularly limited. An example of the seal 144 is a self-sealing seal.

The seal 144 may be arranged in the valve casing 142 to control a flow through the first opening 150A of the sheath 150 and the first opening 108A of the first container outer body 108 into the first interior space 106.

The seal 144 may have a normally closed position that relatively reduces or completely restricts flow through the seal 144. The normally closed position of the seal 144 may be a sealed position of the self-sealing seal.

The seal 144 may be activated from the normally closed position to an activated position. The activated position of the seal 144 allows for relatively increased flow through the seal 144 and the first opening 150A of the sheath 150 and the first opening 108A of the first container outer body 108 into the first interior space 106. For example, the seal 144 may be activated from the normally closed position to the activated position by deforming the self- sealing seal to provide a pathway (see FIG. 24) for flow through the seal 144. The seal 144 may be activated by a valve stem to be described below.

The seal 144 may be made of a resilient material such as rubber, silicone rubber, a flexible plastic or other resilient polymer.

The first container-side valve 140 may further include a valve cap 146 that is configured to be removably connected to the valve casing 142 to removably cover and prevent exposure to or contact with the seal 144.

Referring to FIGS. 20-22, the first container 100 may further include a sheath cap 160 that is removably connected to the sheath 150. The sheath cap 160 may include a sheath cap body 162 that is removably connected to the sheath 150. The sheath 150 and the sheath cap body 162 may be provided with a connection mechanism. For example, an outer surface of the sheath 150 may be provided with a male thread structure and the sheath cap body 162 may be provided with a female thread structure that receives the male thread structure to connect the sheath 150 to the sheath cap 160.

The sheath cap 160 may further include a packing 164 that is a mechanical gasket with a round cross-section. The packing 164 may be seated in a groove in the interior of the sheath cap body 162. Upon connection of the sheath 150 to the sheath cap 160, the packing 164 is compressed between the sheath 150 and the sheath cap body 162 to form a seal at the interface of the sheath 150 and the sheath cap body 162. In this manner, upon connection of the sheath 150 with the sheath cap 160, the first container outer body 108 may be full enclosed within the sheath interior space 152 to prevent exposure of the first container outer body 108 to the environment.

The sheath cap 160 may further include a vent166. The vent 166 (or valve) can be arranged within first interior space 106 to vent displaced air from the first interior space 106 as described in detail below. The sheath cap 160 may further include a filter 168 that permits one way flow of air from the first interior space 106 to prevent exposure of the first container outer body 108 to the environment.

Referring to FIG. 20, the first container 100 may further include a plunger seal 170 arranged within the first interior space 106 of the first container outer body 108 to contact an interior surface of the first container outer body 108. For example, the plunger seal 170 may be inserted from the second opening 108B of the first container outer body 108 into the first interior space 106. The plunger seal 170 is configured to receive a force to move the plunger seal 170 within the first interior space 106 to change a volume of space within the first interior space 106 between the plunger seal 170 and the first container-side valve 140. As the plunger seal 170 is moved towards the first container-side valve 140, the space within the first interior space 106 between the plunger seal 170 and the first container-side valve 140 is decreased. As the plunger seal 170 is moved away from the first container-side valve 140, the space within the first interior space 106 between the plunger seal 170 and the first container-side valve 140 is increased. Further, as the plunger 170 is moved away from the first container-side valve 140, air that is displaced by the plunger 170 out of the second opening 108B can be vented from the vent 166 through the filter 168.

Referring to FIG. 20, the first container 100 may further include a movable body 400. The movable body 400 may be arranged in the space within the first interior space 106 between the plunger seal 170 and the first container-side valve 140. The movable body 400 may have a structure described above with reference to FIGS. 13A-D.

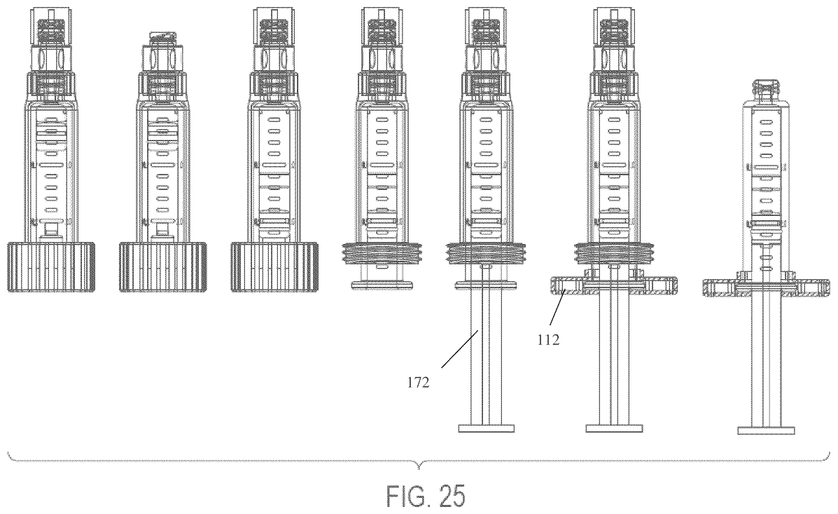

Referring to FIGS. 20 and 25, the first container 100 may further include a plunger 172. The plunger 172 may be removably connected to the plunger seal 170. Upon connection of the plunger 172 to the plunger seal 170, the plunger 172 is configured to transfer a force to the plunger seal 170 to move the plunger seal 170 within the first interior space 106 of the first container outer body 108.

Referring to FIGS. 20 and 25, the first container 100 may further include a first container outer body flange 110. The first container outer body flange 110 may be detachably attached to the first container outer body 108. The first container outer body flange 110 may be configured to support the fingers of a user or to be fitted withing a syringe pump. Pressure exerted between the plunger 172 and the first container outer body flange 110 results in the application of force to the plunger seal 170 to move the plunger seal 170 within the first interior space 106 of the first container outer body 108.

### Second Container (Wash Tube)

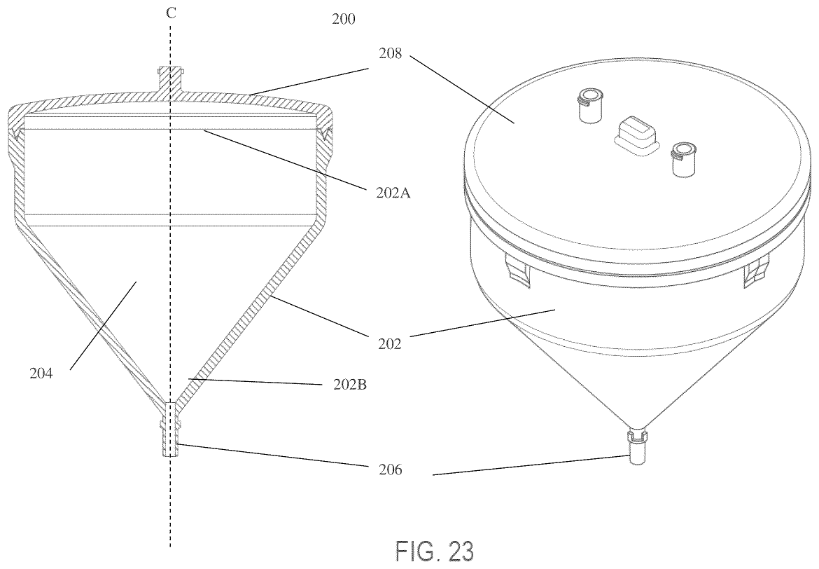

The second container 200 will now be described with reference to FIGS. 23 and 24.

The second container 200 may include a second container outer body 202, a second container-side stem 204 and a second container cover 206.

The second container outer body 202 may define a second interior space 204. The second interior space 204 may be connected to a first opening 202A at a first end of the second container outer body 202 and a second opening 202B at a second end of the second container outer body 202. The first opening 202A and the second opening 202B may be provided along a second container outer body axis C.

The volume of the second container outer body 202 is not particularly limited. For example, the volume of the second interior space 204 may be selected based on a target dose volume of a dose to be prepared. The volume of the second interior space 204 can be, for example, about 50 mL or about 300 mL

The second container cover 208 may be provided to be removably connected to the second container outer body 202 to cover the first opening 202A of the second container 200. Further, the second container-side stem 206 may be provided to be connected to the second container outer body 202 at the second opening 202B of the second container outer body 202 or may be formed integrally with the second container outer body 202 to provide the second opening 202B of the second container outer body 202.

An interior surface of the second container outer body 202 that defines the second interior space 204 can have a part having a conical shape. The conical shape can have a varying diameter that reduces along the second container outer body axis C in a direction from the first opening 202A of the second container outer body 202 towards the second opening 202B of the second container outer body 202. The conical shape of the second interior space 204 may assist in channeling a flow of fluid towards the second container-side stem 206, as described below.

A material or materials for forming the second container outer body 202, the second container-side stem 206 and the second container cover 208 is not particularly limited. Examples of the material or materials for forming the second container outer body 202, the second container-side stem 206 and the second container cover 208 may include low leachable and/or extractable materials such as cyclic olefin polymer, cyclic olefin copolymer, perfluoroalkoxy alkane, ethylene tetra fluoroethylene, cyclic block copolymers, polyethylene, polypropylene and polycarbonates. The material or materials for forming the second container outer body 202 may also be selected based on contemplated methods or manufacture, such as injection molding.

Referring to FIG. 24, the system 10 can further include a connector 210 configured to removably connect the second container 200 to the first container 100.

Upon connection of the second container 200 to the first container 100 via the connector 210, the second container-side stem 206 is arranged at a position to activate the first container-side valve 140 from the normally closed position to the activated position to relatively increase flow through the seal 144 by communicating the second interior space 204 and the first interior space 106. For example, as shown in FIG. 24, the second container-side stem 206 is positioned by the connector 210 to bias the seal 144 of the first container-side valve 140 from the normally closed position to the activated position to communicate first interior space 106 of the first container outer body 108 with the second interior space 204 of the second container 200. Process for Preparing a Cell Solution for Delivery

A process for preparing a cell solution for delivery will be described with reference to FIGS. 24 and 25. The process may include a sequence of steps involving configurations of the first container 100 as shown from the left to right side of FIG. 25.

The process for preparing the cell solution for delivery may include a step of providing the first container 100 as shown in FIG. 25. The first container 100 may be in an assembled state in which the first container outer body 108 is at least partially arranged in the sheath interior space 152, with the first container-side valve 140 attached to the first opening 108A of the first container outer body 108, the valve cap 146 connected to the first container-side valve 140 and the sheath cap 160 connected to the sheath 150. The first container 100 may be pre-filled with a cell delivery solution. In this assembled state, contamination of the first container outer body 108 and in particular the first interior space 106 can be minimized. The first container can also be transported to a site for preparing the cell solution for delivery in this assembled state.

The process for preparing the cell solution for delivery may then proceed to a step of connecting the second container 200 to the first container as shown in FIG. 24. In this step, the valve cap 146 may be disconnected from the valve casing 142 to expose the seal 144 of the first container-side valve 140. Further, the second container 200 may be connected by the intermediate structure to the first container 100 such that the second container-side stem 206 contacts the seal 144 to bias the seal 144 from the normally closed position to the activated position to relatively increase flow through the seal 144 by communicating the second interior space 204 and the first interior space 106.

The process for preparing the cell solution for delivery may then proceed to a step of introducing or providing a mixture of wash solution, cells and cryoprotectant into the second interior space 204 of the second container 200. The mixture can be directly introduced or provided to the second interior space 204, or can be introduced through one or more openings provided in the second container cover 208.

The process for preparing the cell solution for delivery may then proceed to a step of applying a centrifugal force to the connected first container 100 and the second container 200. This step may including loading the connected first container 100 and the second container 200 into a centrifuge in an orientation such that during the spinning operation of the centrifuge along a spin axis, at least a portion of the cells in the second container 200 moves outward in the radial direction of the spin axis from the second interior space 204 of the second container 200 to the first interior space 106 of the first container 100 through the second container-side stem 206 and the seal 144 of the first container-side valve 140 activated by the container-side stem 206 from the normally closed position to the activated position. The portion of the cells that is moved by centrifugation from the second container 200 to the first interior space 106 of the first container 100 becomes part of the cell solution for delivery that is prepared and arranged in the first interior space 106 of the first container 100. As the cells are moved from the second container 200 to the first interior space 106, the plunger 170 is moved away from the first container-side valve 140 and air that is displaced by the plunger 170 out of the second opening 108B can be vented from the vent 166 through the filter 168.

The process for preparing the cell solution for delivery may then proceed to a step of removing the first container 100 and the second container 200 from the centrifuge, disconnecting the second container 200 from the first container 100, and connecting the valve cap 146 to the valve casing 142 to cover seal 144 to prevent contamination via the first container-side valve 140 of the cell solution for delivery provided in the first interior space 106.

Further, a mixer that may be optionally provided in the first interior space 106 of the first container 100 may be operated in the manner described above to mix the contents of a first container (e.g., a cell delivery solution, or a cell solution for delivery).

The process for preparing the cell solution for delivery may then proceed to a step of disconnecting the sheath cap 160 from the sheath 150, as shown in FIG. 25.

The process for preparing the cell solution for delivery may then proceed to a step of connecting the plunger 172 to the plunger seal 170 and connecting the first container outer body flange 110 to the first container outer body 108, as shown in FIG. 25.

The process for preparing the cell solution for delivery may then proceed to a step of withdrawing the first container outer body 108 from the sheath interior space 152 of the sheath 150. Upon withdrawal of the first container outer body 108 from the sheath interior space 152 of the sheath 150, the first container outer body 108 is disconnected from the first container-side valve 140, as show in FIG. 25. This step may be performed in a clean (e.g., aseptic) environment to minimize contamination of the cell solution for delivery via the first opening 108A of the first container outer body 108.

At any point after the at least a portion of the cells is moved by the centrifugal force into the first interior space 106 of the first container 100, a step of moving the movable body 400 within the first interior space 106 to mix the cell solution for delivery to even disperse the cells can be performed. This step can be repeated as needed.

Upon completion of the above-described steps, the cell solution for delivery is prepared and loaded into the first interior space 106 of the first container 100. The cell solution for delivery and can then be delivered to a subject. For example, the first container 100 can be held manually or fitted within a syringe pump. Pressure can then be exerted between the plunger 172 and the first container outer body flange 110 resulting in the application of force to the plunger seal 170 to move the plunger seal 170 within the interior space 106 to eject the cell solution for delivery from the interior space 106 via the first opening 108A. The ejected cell solution for delivery can be delivered to a subject via a needle directly or indirectly connected to the first opening 108A of the first container 100.

In some embodiments, a device as disclosed herein has some or all of the features shown in FIGS. 20-24 and optional FIG. 25.

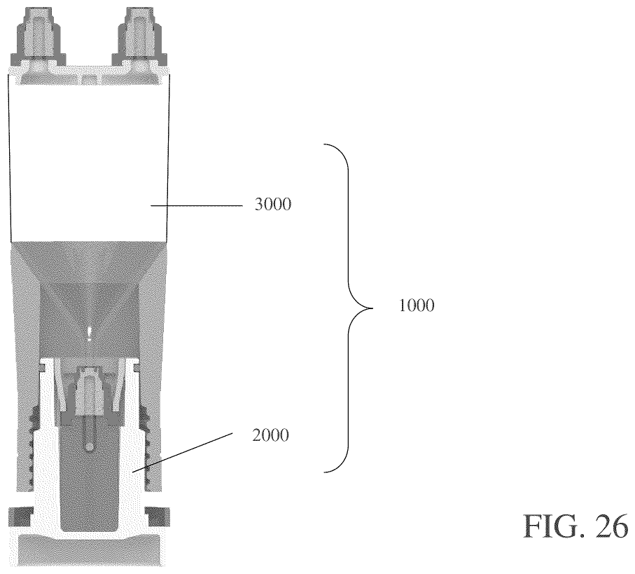

FIG. 26 illustrates an example of a medical device system 1000 for implementing dose preparation according to one embodiment. The medical device system 1000 may include a first container 2000 (also referred to as a dose preparation tube) and a second container 3000 (also referred to as a wash tube) that is detachably connected to the first container 2000.

### First Container (Dose Preparation Tube)

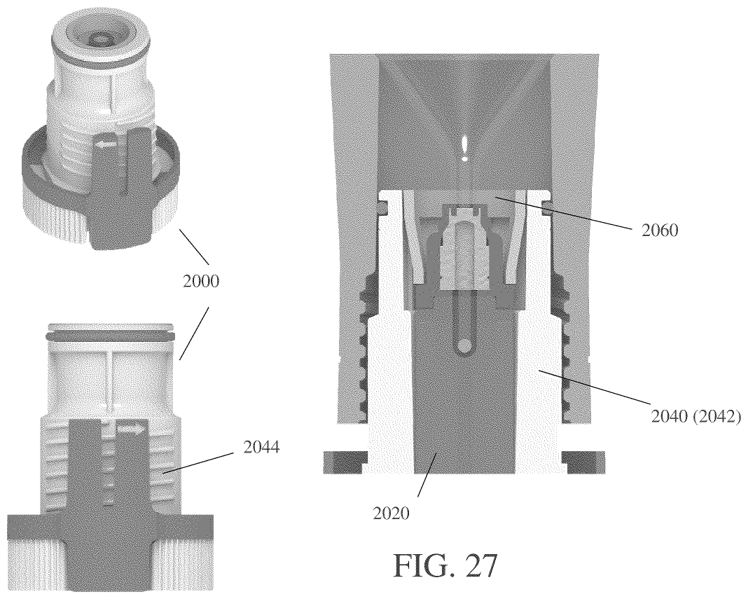

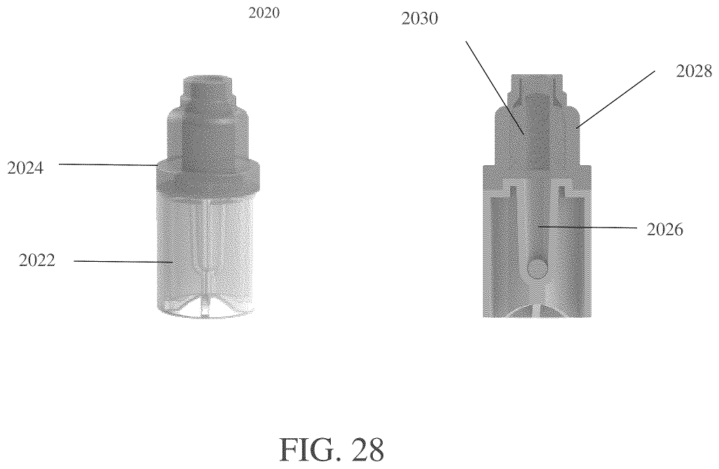

The first container 2000, that is an apparatus, will now be described with reference to FIGS. 27 and 28. The first container 2000 may include a first container tube 2020, a sheath 2040 and a retention structure 2060.

The first container tube 2020 may include a first container tube body 2022 and a first container-side valve 2024 attached to the first container body 2022. The first container-side valve 2024 and the first container body 2022 define a first interior space 2026 extending in a longitudinal axis of the first container tube 2020. The first container-side valve 2024 may include a first container-side valve body 2028 attached to the first container body 2022 and a first container-side valve seal 2030 arranged within the first container-side valve body 2028. The first container-side valve body 2028 defines a first opening of the first container 2000 that communicates with the first interior space 2026.

The first container-side valve seal 2030 is formed to be in a normally closed position that relatively reduces flow through the first container-side valve 2024 into the first interior space 2026. For example, in the normally closed position, the first container-side valve seal 2030 can prevent or block flow through the first opening of the first container 2000 into the first interior space 2026. The first container-side valve seal 2030 is further formed to be activated, upon connection of the first container 2000 to the second container 3000, from the normally closed position to an activated position that relatively increases flow through the first opening of the first container 2000 into the first interior space 2026. Activation of the first container-side valve 2024 will be described in additional detail below.

Although the first container-side valve 2024 is described as including a valve body and a valve seal, the first container-side valve 2024 can also include other structures that restrict flow into the first interior space 2026 and that is activated upon connection of the first container 2000 and the second container 3000. For example, the first container-side valve 2024 can include a valve body and a membrane (or barrier) that blocks flow into the first interior space 2026 and that is activated by perforating the membrane to allow flow into the first interior space 2026.

The first container 2000 may also include a movable body such as the movable bodies shown in FIGS. 13A-13D.

Returning to FIG. 27, the sheath 2040 and the retention structure 2060 will be described in detail.

The sheath 2040 can include a sheath body 2042 that defines an interior space and an opening leading into the interior space. The interior space of the sheath body 2042 may be dimensioned to receive the first container tube 2020.

The retention structure 2060 can be attached to the sheath body 2042 to retain the first container tube 2020 within the interior space of the sheath body 2042. The retention structure 2060 can be a collar, as shown in FIG. 27, that is provided at the opening leading into the interior space of the sheath body 2042. The collar can include a flexible tapered structure that narrows as the collar extends along the longitudinal axis of the first container 2000 away from the opening of the sheath body 2042 towards the interior space of the sheath body 2042. As the first container tube 2020 is moved past the opening of the sheath body 2042 towards the interior space of the sheath body 2042, the first container tube 2020 flexibly spreads the collar in a radial direction of the longitudinal axis of the first container 2000 to allow the first container tube 2020 to be moved into and arranged within the interior space of the sheath body 2042. In a position where the first container tube 2020 is arranged within the interior space of the sheath body 2042, the collar then returns to its tapered form to retain the first container tube 2020 within the interior space of the sheath body 2042. For example, the tapered form of the collar abuts against the first container tube 2020 to prevent movement of first container tube 2020 out of the interior space of the sheath body 2042 towards the opening of the sheath body 2042 along the longitudinal axis of the first container 2000.

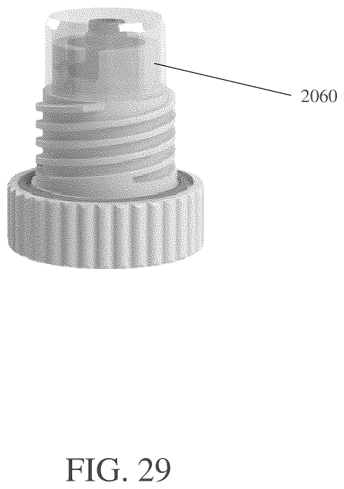

The retention structure 2060 is not limited to the collar shown in FIG. 27. For example, the retention structure 2060 can include a retention cap as shown in FIG. 29. The retention cap can be removably attached to the sheath body 2042 to enclose the first container tube 2020 within the interior space of the sheath body 2042. In FIG. 29, the retention cap and the sheath body 2042 are shown as being removably attached by a bayonet connection.

Further, the sheath 2040 can include a sheath body connection mechanism 2044 that is configured to connect the first container 2000 to the second container 3000, as described in detail below. As an example, the sheath body connection mechanism 2044 can include one of a male thread or female thread that connects to a corresponding female thread or male thread provided to the second container 3000.

### Second Container (Wash Tube)

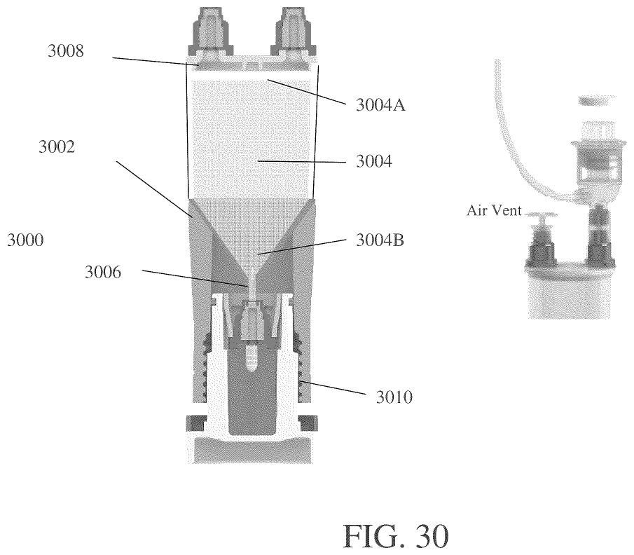

The second container 3000, that is an apparatus, will now be described with reference to FIG. 30.

The second container 3000 may include a second container outer body 3002 that extends along a longitudinal axis of the second container 3000. The second container outer body 3002 defines a second interior space 3004 having a first opening 3004A and a second opening 3004B at two ends of the second interior space 3004 along the longitudinal axis.

The volume of the second container outer body 202 is not particularly limited. For example, the volume of the second interior space 204 may be selected based on a target dose volume of a dose to be prepared.

A material or materials for forming the second container outer body 202, the second container-side stem 206 and the second container cover 208 is not particularly limited. Examples of the material or materials for forming the second container outer body 202, the second container-side stem 206 and the second container cover 208 may include resins such as polypropylene and polycarbonates.

The second container 3000 may include a second container-side stem 3006 at the second opening 3004B

The second container 3000 may include a second container cover 3008 that is configured to be removably connected to the second container outer body 3002 to cover the first opening 3004A of the second container outer body 3002.

The second container cover 3008 may define one or more openings for accessing the second interior space 3004. For example, the second container cover 3008 may define a first opening through which a mixture of wash solution, cells and cytoprotectant may be provided into the second interior space 3004. The second container cover 3008 may further define a second opening through which air that is displaced by the mixture of wash solution, cells and cryoprotectant may be vented from the second interior space 3004. The second opening may also provide access to sample the mixture provided into the second interior space 3004 and adjust the volume and/or the concentration of the mixture provided into the second interior space 3004.

An interior surface of the second container outer body 3002 that defines the second interior space 3004 can have a part having a conical shape. The conical shape can have a varying diameter that reduces along the longitudinal axis of the second container 3000 in a direction from the first opening 3004A of the second container outer body 3002 towards the second opening 3004B of the second container outer body 3002. The conical shape of the second interior space 3004 may assist in channeling a flow of fluids towards the second container-side stem 3006, as described below.

The second container 3000 may further include a second container connection mechanism 3010 configured to connect the second container 3000 to the first container 2000. In an example, the second container connection mechanism 3010 can include a tubular extension extending from the second container outer body 3002 and away from the second container-side stem 3006. An interior surface of the tubular extension may be provided with the corresponding female thread or male thread that mates with the male thread or female thread of the sheath body connection mechanism 2044 to connect the second container 3000 to the first container 2000.

Although a screw-type connection for connecting the first container 2000 and the second container 3000 is described, other types of connections can be provided. For example, a ratcheted connection allowing for the first container 2000 to be pushed towards the container 3000 in a step-wise manner can be provided.

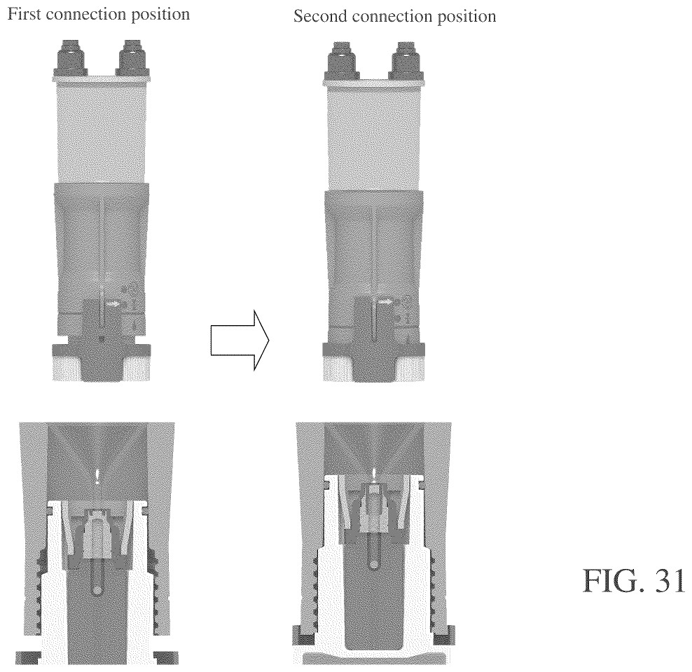

FIG. 31 illustrates two connection positions of the first container 2000 and the second container 3000. In a first connection position, the first container 2000 is connected to the second container 3000 by the sheath body connection mechanism 2044 and the second container connection mechanism 3010 by for example rotation of the first container 2000 relative to the second container 3000 such that the first container-side valve seal 2030 abuts the second container-side stem 3006. By positioning the first container side valve seal 2030 to abut the second container-side stem 3006, flow from the second interior space 3004 through the second container-side stem 3006 can be reduced or prevented.

In a second connection position, the first container 2000 is connected to the second container 3000 by the sheath body connection mechanism 2044 and the second container connection mechanism 3010 by for example additional rotation of the first container 2000 relative to the second container 3000 such that the second container-side stem 3006 is moved past its position in the first connection position to penetrate the first container-side valve seal 2030 to thereby activate the first container-side valve 2024 from the normally closed position to the activated position. In the second connection position, the second interior space 3004 of the second container 3000 is communicated with the first interior space 2026 of the first container 2000 to allow flow from the second interior space 3004 to the first interior space via the second container-side stem 3006.

In some embodiments, a device as disclosed herein has some or all of the features shown in FIGS. 26-34 with an optional magnetic system to move a mixer operated by hand or automatically by an accessory device.

### Sheath Cap

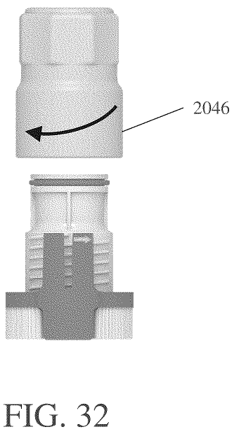

Referring to FIG. 32, the medical device system 1000 may include a sheath cap 2046. The sheath cap 2046 can be removably connected to the sheath 2040 to enclose the first container tube 2020 within the interior space of the sheath 2040. In an example, the sheath cap 2046 may be provided with corresponding female threads or male threads that connect the sheath cap 2046 to the male threads or the female threads of the sheath body connection mechanism 2044 of the sheath 2040. The sheath cap 2046 can be connected to the sheath 2040 to separate the first container tube 2020 from an external environment.

### Loading Adapter

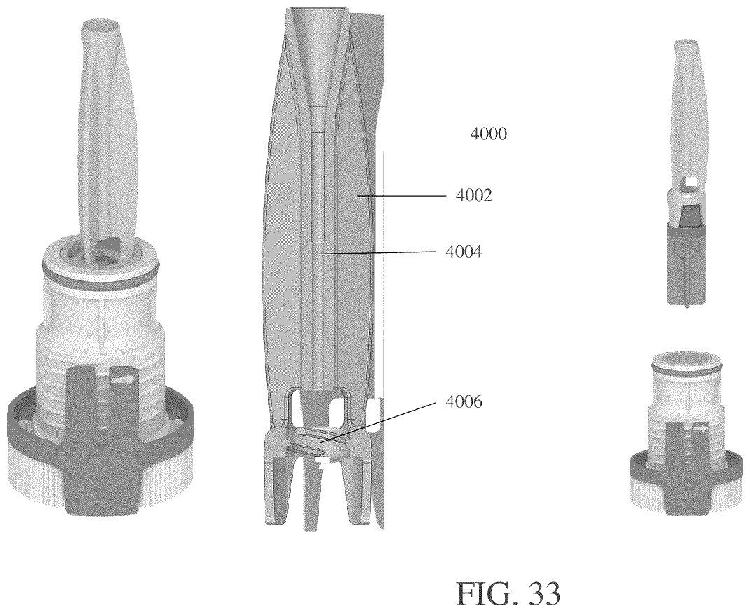

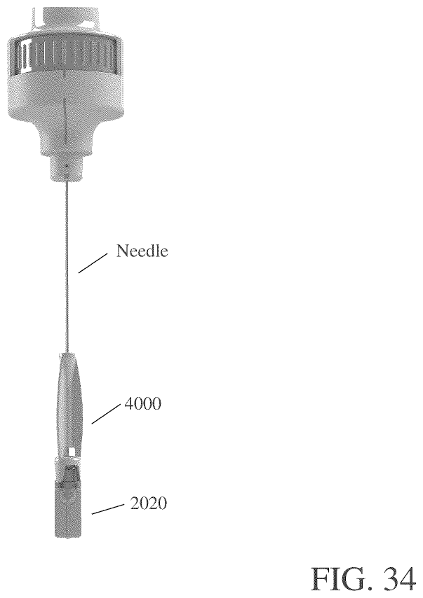

Referring to FIGS. 33 and 34, the medical device system 1000 may include a loading adapter 4000. The loading adapter 4000 may include a loading adapter body 4002 defining a guide space 4004 that extends along a longitudinal axis of the loading adapter body 4002. Further, loading adapter 4000 may include a loading adapter connector 4006.

In an example, the loading adapter connector 4006 can be provided at one end of the loading adapter body 4002 along the longitudinal axis of the loading adapter body 4002. The loading adapter connector 4006 can be configured to be detachably connected to the first container tube 2020 and to separate the first container tube 2020 from the sheath 2040.

In an example, the loading adapter connector 4006 can include a connection mechanism such as a male thread or female thread that can be detachably connected to a corresponding female thread or male thread provided on an exterior surface of the first container-side valve body 2028. Further, the loading adapter connector 4006 can include one or more fingers. In operation, the longitudinal axis of the loading adapter body 4002 is aligned with the longitudinal axis of the sheath body 2042. The loading adapter connector 4006 is then arranged within the collar to flexibly spread the collar in the radial direction of the longitudinal axis of the first container 2000. The loading adapter 4000 is then rotated along the longitudinal axis of the loading adapter 4000 to mate the male thread or female thread of the loading adapter connector 4006 to the corresponding female thread or male thread of the first container-side valve body 2028 to connect the loading adapter 4000 to the first container tube 2020. The loading adapter 4000 is then separated from the sheath 2040 while still spreading the collar in the radial direction of the longitudinal axis of the first container 2000 to facilitate withdrawal of the first container tube 2020 from the interior space of the sheath body 2042.

Referring to FIG. 34, in an arrangement where the loading adapter 4000 is connected to the first container tube 2020, the guide space 4004 of the loading adapter 4000 may guide a distal end of a device to the first container-side valve 2024 to facilitate transfer of a dose prepared within the first interior space 2026 to the device. The distal end of the device may include a needle or a tip of a pipette. The guide space 4004 may serve protect the needle from damage as the needle approaches the first container 2000. Further, the guide space 4004 may serve to more accurately guide the needle towards a designated point in the first container-side valve seal 2030.

### Process for Preparing a Cell Solution for Delivery

A process for preparing a cell solution for delivery will be described with reference to FIGS. 26-34.

The process for preparing the cell solution for delivery may include a step of providing the first container 2000, as shown in FIG. 28, retained by the collar within sheath 2040, as shown in FIG. 27. The first container 2000 may be pre-filled with a cell delivery solution.

The process for preparing the cell solution for delivery may further include a step of connecting the first container 2000 to the second container 3000 such that the first container-side valve seal 2030 abuts the second container-side stem 3006 in the first connection position shown in FIG. 31. By positioning the first container side valve seal 2030 to abut the second container-side stem 3006, flow from the second interior space 3004 through the second container-side stem 3006 can be reduced or prevented.

The process for preparing the cell solution for delivery may further include a step of introducing or providing a mixture of wash solution, cells and cryoprotectant into the second interior space 3004 of the second container 3000.

The process for preparing the cell solution for delivery may further include a step of arranging the first container 2000 and the second container 3000 in the second connection position shown in FIG. 31 by for example rotation of the first container 2000 relative to the second container 3000 such that the second container-side stem 3006 is moved past its position in the first connection position to penetrate the first container-side valve seal 2030 to thereby activate the first container-side valve 2024 from the normally closed position to the activated position. In the second connection position, the second interior space 3004 of the second container 3000 is communicated with the first interior space 2026 of the first container 2000 to allow flow from the second interior space 3004 to the first interior space 2026 via the second container-side stem 3006.

The process for preparing the cell solution for delivery may further include a step of applying a centrifugal force to the connected first container 2000 and the second container 3000. This step may including loading the connected first container 2000 and the second container 3000 into a centrifuge in an orientation such that during the spinning operation of the centrifuge along a spin axis, at least a portion of the cells in the second container 3000 moves outward in the radial direction of the spin axis from the second interior space 3004 of the second container 3000 to the first interior space 2026 of the first container 2000 through the second container-side stem 3006 and the seal 2030 of the first container-side valve 2024 activated by the second container-side stem 3006. The portion of the cells that is moved by centrifugation from the second container 3000 to the first interior space 2026 of the first container 2000 becomes part of the cell solution for delivery that is prepared and arranged in the first interior space 2026 of the first container 2000.

The process for preparing the cell solution for delivery may further include a step of disconnecting the first container 2000 from the second container 3000, and connecting the sheath cap 2046 to the sheath 2040 to enclose the first container tube 2020 within the interior space of the sheath 2040. In this state, the first container tube 2020 having the cell solution for delivery contained therein can be stored for later use or for transportation. Further, a mixer that may be optionally provided in the first interior space 2026 of the first container 2000 may be operated in the manner described above to mix the cell solution for delivery.

The process for preparing the cell solution for delivery may further include a step of disconnecting the sheath cap 2046 from the sheath 2040 and using the loading adapter 4000 to withdraw the first container tube 2020 from the interior space of the sheath body 2042.

The process for preparing the cell solution for delivery may further include a step of guiding a needle or pipette through the guide space 4004 of the loading adapter 4000 to penetrate the first container-side valve seal 2030 whereby a portion of the cell solution for delivery can be aspirated into the needle or pipette.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements, if any, in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1.-107. (canceled)

108. A medical device system comprising:
a first container; and
a second container,
wherein the first container comprises:
a first container outer body, wherein the first container outer body defines a first interior space connected to a first opening of the first container; and
a first container-side valve configured to be activated, upon connection of the second container to the first container, from a normally closed position that relatively reduces flow through the first opening of the first container into the first interior space to an activated position that relatively increases flow through the first opening of the first container into the first interior space, and
wherein the second container comprises:
a second container outer body that defines a second container interior space; and
a stem fluidly connected to the second container interior space,
wherein, upon connection of the second container to the first container, the first container-side valve is configured to be brought into contact with the stem and the stem is configured to activate the first container-side valve from the normally closed position to the activated position.

109. The medical device system according to claim 108,
wherein the second container interior space extends along a longitudinal direction,
wherein at a first end portion of the second container interior space along the longitudinal direction, a width of the second container interior space decreases towards a first end opening of the second container interior space, and
wherein the stem is fluidly connected to the first end opening of the second container interior space and extends along the longitudinal direction.

110. The medical device system according to claim 109,
wherein the stem is connected to the second container outer body to be stationary relative to the second container interior space.

111. The medical device system according to claim 108,
wherein the first container further comprises a sheath defining a sheath interior space, and
wherein at least a portion of the first container outer body is configured to be removably inserted within the sheath interior space.

112. The medical device system according to claim 111,
wherein the sheath is configured to protect the contents of the first container from contamination and the first container is at least partially insertable within the sheath to protect the contents of the first container from contamination.

113. The medical device system according to claim 111,
wherein the first container-side valve is configured to be connected to a first end portion of the sheath, and
wherein the first container outer body is configured to be arranged within the sheath interior space to contact the first container-side valve such that upon activation of the first container-side valve from the normally closed position to the activated position the flow through the first opening of the first container into the first interior space is relatively increased.

114. The medical device system according to claim 113,
wherein the first container further comprises a sheath cap configured to be removably connected to a second end portion of the sheath with the first container outer body being enclosed within the sheath by the sheath cap.

115. The medical device system according to claim 114,
wherein the sheath cap comprises:
a vent configured to vent air displaced from first interior space; and
a filter arranged within the vent.

116. The medical device system according to claim 115,
wherein the sheath cap defines a through hole through which gas in the interior space of the first container outer body is vented.

117. The medical device system according to claim 108,
wherein the first container further comprises a valve cap configured to be removably connected to the first container-side valve to prevent direct contact of the first container-side valve from an exterior of the valve cap.

118. The medical device system according to claim 108,
wherein the first container further comprises a plunger seal arranged within the first interior space, wherein the plunger seal is configured to receive a force to move the plunger seal within the first interior space to change a volume of space within the first interior space between the plunger seal and the first container-side valve.

119. The medical device system according to claim 118, further comprising a plunger,
wherein the plunger is configured to be removably connected to the plunger seal.

120. The medical device system according to claim 118, further comprising a plunger configured to apply the force to the plunger seal to move the plunger seal within the first interior space to change the volume of space within the first interior space between the plunger seal and the first container-side valve.

121. The medical device system according to claim 108, further comprising:
a movable body arranged within the first interior space of the first container outer body and configured to move within the first interior space.

122. The medical device system according to claim 121,
wherein the movable body comprises a stainless-steel body.

123. The medical device system according to claim 121,
wherein the movable body is configured to be moved within the first interior space by a magnetic force exerted from outside of the first container.

124. The medical device system according to claim 121,
wherein the movable body defines one or more through holes connecting a first side of the movable body facing the first container-side valve and a second side of the movable body opposite to the first side of the movable body.

125. An apparatus comprising:
a first container for connecting to a second container,
wherein the second container comprises:
a second container outer body that defines a second container interior space; and
a stem fluidly connected to the second container interior space, and
wherein the first container comprises:
a first container outer body, wherein the first container outer body defines a first interior space connected to a first opening of the first container; and
a first container-side valve configured to, upon connection of the second container to the first container, be brought into contact with the stem to be activated from a normally closed position that relatively reduces flow through the first opening of the first container into the first interior space to an activated position that relatively increases flow through the first opening of the first container into the first interior space.

126. The apparatus according to claim 125,
wherein the first container further comprises a plunger seal arranged within the first interior space, wherein the plunger seal is configured to receive a force to move the plunger seal within the first interior space to change a volume of space within the first interior space between the plunger seal and the first container-side valve.

127. The apparatus according to claim 126,
wherein the plunger is configured to be removably connected to the plunger seal.

128. The apparatus according to claim 126, further comprising a plunger configured to apply the force to the plunger seal to move the plunger seal within the first interior space to change the volume of space within the first interior space between the plunger seal and the first container-side valve.

129. The apparatus according to claim 125,
wherein the first container further comprises a sheath defining a sheath interior space, and
wherein at least a portion of the first container outer body is configured to be removably inserted within the sheath interior space.

130. The apparatus according to claim 129,
wherein the first container-side valve is configured to be connected to a first end portion of the sheath, and
wherein the first container outer body is configured to be arranged within the sheath interior space to contact the first container-side valve such that upon activation of the first container-side valve from the normally closed position to the activated position the flow through the first opening of the first container into the first interior space is relatively increased.

131. The apparatus according to claim 130,
wherein the first container further comprises a sheath cap configured to be removably connected to a second end portion of the sheath with the first container outer body being enclosed within the sheath by the sheath cap.

132. The apparatus according to claim 130,
wherein the sheath cap comprises:
a vent configured to vent air displaced from first interior space; and
a filter arranged within the vent.

133. A process for operating a medical device system comprising:
a first container; and
a second container,
wherein the first container comprises:
a first container outer body;
a first container barrel, arranged within the first container outer body, that defines a first interior space connected to a first opening of the first container and a second opening of the first container;
a first container-side valve; and
a plunger seal arranged within the first container barrel between the first opening,
wherein the process comprises:
aligning the first container and the second container;
connecting the second container to the first container outer body, wherein the first container-side valve is configured to be activated, upon connection of the second container to the first container outer body, from a normally closed position that relatively reduces flow through the first opening of the first container into the first interior space to an activated position that relatively increases flow through the first opening of the first container into the first interior space; and
applying a force to move the plunger seal within the first container barrel to change a volume of space within the first interior space between the first opening and the plunger seal.
